(19) United States
(12) Patent Application Publication
(10) Pub. No.: US 2021/0275498 A1
(43) Pub. Date: Sep. 9, 2021

(54) IMIDAZOLYL ETHANAMIDE PENTANDIOIC ACID FOR USE IN THERAPY OF SYMPTOMS RELATED TO EXPOSURE TO LETHAL RADIATION

(71) Applicant: MYELO THERAPEUTICS GMBH, Berlin (DE)

(72) Inventor: Dirk PLEIMES, Berlin (DE)

(73) Assignee: MYELO THERAPEUTICS GMBH, Berlin (DE)

(21) Appl. No.: 17/279,625

(22) PCT Filed: Sep. 25, 2019
(86) PCT No.: PCT/EP2019/075853

§ 371 Date: Mar. 25, 2021

(30) Foreign Application Priority Data
Sep. 25, 2018 (EP) 18196490.9
Mar. 20, 2019 (EP) 19164201.6

(51) Int. Cl. A61K 31/417; A61K 47/60; A61K 38/19; A61P 43/00

(52) U.S. Cl. CPC A61K 31/417; A61P 43/00; A61K 38/193; A61K 47/60

(57) ABSTRACT

The present invention relates to the use of imidazolyl ethanamide pentandioic acid for prevention or treatment of radiation-induced damage. The invention further relates to a combination medicament for use in treatment or prevention of radiation-induced damage comprising imidazolyl ethanamide pentandioic acid combined with G-CSF or GM-CSF.

The present invention relates to the use of imidazolyl ethanamide pentandioic acid for treatment or prevention of radiation-induced damage.

### DESCRIPTION

Humans and animals are highly susceptible to radiation-induced damage resulting in cellular, tissue, organ and systemic injuries. In accidental radiation exposure, such as a nuclear explosion or a disaster scenario, many victims will suffer from Acute Radiation Syndrome (ARS) to varying degrees. The immediate objectives at a radiation disaster scene are quite different from the radiation treatment of cancer. In such disaster scenario, early efforts involve reaching as many afflicted individuals as possible with a treatment that could prolong life, so that victims can be successfully triaged and receive subsequent, in-depth medical care as dictated by their individual condition. Another aspect of radiation disaster management is that any life-saving drugs or treatments need to be available at protracted time points following the radiation disaster. This requirement is due to the time needed to mobilize medical staff, drugs/treatments, and equipment to a disaster scene, so that life-saving drugs/treatments could be administered to the victims. FDA requires medical countermeasures to be effective when administered not later than 24 h after radiation exposure.

In addition to incidental radiation exposure due to a disaster, radiation-induced damage to cells, tissues, organs and systems can be the result of radiation exposure in the course of a treatment for a disease, such as cancer. Over 40% of cancer patients will require radiation therapy during management of their disease. Although radiation therapy improves the survival of a significant number of cancer patients, both acute radiation toxicity (which manifests itself during a course of clinical radiotherapy or shortly thereafter), and late toxicity (developing months to years after completion of radio therapy) compromise overall outcomes for successfully treated cancer patients.

Currently, there are agents that can protect cells and tissues from radiation treatments used in cancer, such as colony stimulating factors (CSFs). In terms of accidental or intentional radiation exposure, there are three medical countermeasures approved by FDA that showed increased survival in mice and NHPs after total body irradiation. However, all three drugs are administered by subcutaneous injection, which might be inconvenient in case of mass casualty scenario.

Based on the above-mentioned state of the art, the objective of the present invention is to provide means and methods to treat or prevent radiation-induced damage in a human subject. This objective is attained by the subject-matter of the independent claims of the present specification.

### Terms and Definitions

The term imidazolyl ethanamide pentandioic acid in the context of the present specification relates to 5-{[2-(1H-imidazol-4-yl)ethyl]amino}-5-oxo-pentanoic acid (CAS number 219694-63-0). The term Myelo001 is a synonym for imidazolyl ethanaide pentandioic acid.

The term G-CSF in the context of the present specification relates to granulocyte-colony stimulating factor.

The term GM-CSF in the context of the present specification relates to granulocyte-macrophage colony stimulating factor.

The term peg-G-CSF or peg-GM-CSF in the context of the present specification relates to pegylated G-CSF or GM-CSF. Pegylation relates to modification with polyethylene glycol.

The term lipeg-G-CSF in the context of the present specification relates to granulocyte-colony stimulating factor covalently linked with a single methoxy PEG molecule via a carbohydrate linker consisting of glycine, N-acetylneuraminic acid and N-acetylgalactosamine.

As used herein, the term treating or treatment of any disease or disorder (e.g. cancer) refers in one embodiment, to ameliorating the disease or disorder (e.g. slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. Treatment also refers to application after the triggering event of the disease, disorder or damage. Methods for assessing treatment and/or prevention of disease are generally known in the art, unless specifically described herein below.

The skilled person is aware that any specifically mentioned drug may be present as a pharmaceutically acceptable salt of said drug. Pharmaceutically acceptable salts comprise the ionized drug and an oppositely charged counterion. Non-limiting examples of pharmaceutically acceptable anionic salt forms include acetate, benzoate, besylate, bitatrate, bromide, carbonate, chloride, citrate, edetate, edisylate, embonate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate, phosphate, diphosphate, salicylate, disalicylate, stearate, succinate, sulfate, tartrate, tosylate, triethiodide and valerate. Non-limiting examples of pharmaceutically acceptable cationic salt forms include aluminium, benzathine, calcium, ethylene diamine, lysine, magnesium, meglumine, potassium, procaine, sodium, tromethamine and zinc.

Dosage forms may be for enteral administration, such as nasal, buccal, rectal, transdermal or oral administration, or as an inhalation form or suppository. Alternatively, parenteral administration may be used, such as subcutaneous, intravenous, intrahepatic or intramuscular injection forms. Optionally, a pharmaceutically acceptable carrier and/or excipient may be present.

### DETAILED DESCRIPTION OF THE INVENTION

A first aspect of the invention relates to imidazolyl ethanamide pentandioic acid for use in treatment or prevention of radiation-induced damage.

In certain embodiments, imidazolyl ethanamide pentandioic acid is administered to a human patient at a dose of 0.4 mg/kg to 12 mg/kg b.w. In certain embodiments, imidazolyl ethanamide pentandioic acid is administered to a human patient at a dose of 1.2 mg/kg to 12 mg/kg b.w. In certain embodiments, imidazolyl ethanamide pentandioic acid is administered to a human patient at a dose of 4 mg/kg to 12 mg/kg b.w.

In certain embodiments, imidazolyl ethanamide pentandioic acid is administered to a patient of age 2 to 16 at a dose of 0.5 mg/kg to 3.75 mg/kg b.w. In certain embodiments, imidazolyl ethanamide pentandioic acid is administered to a patient of age 2 to 16 at a dose of 1.25 mg/kg to 3.25 mg/kg b.w. In certain embodiments, imidazolyl ethanamide pentandioic acid is administered to a patient of age 2 to 16 at a dose of 1.25 mg/kg b.w. twice a day.

In certain embodiments, imidazolyl ethanamide pentandioic acid is administered at 1 h to 120 h before radiation exposure. In certain embodiments, imidazolyl ethanamide pentandioic acid is administered at 1 h to 120 h before radiation exposure. In certain embodiments, imidazolyl ethanamide pentandioic acid is administered at 1 h to 72 h before radiation exposure. In certain embodiments, imidazolyl ethanamide pentandioic acid is administered at 6 h to 48 h before radiation exposure. In certain embodiments, imidazolyl ethanamide pentandioic acid is administered at 12 h to 24 h before radiation exposure.

In certain embodiments, a first dose of imidazolyl ethanamide pentandioic acid is administered at 24 h to 120 h after radiation exposure. In certain embodiments, a first dose of imidazolyl ethanamide pentandioic acid is administered at 24 h to 72 h after radiation exposure. In certain embodiments, a first dose of imidazolyl ethanamide pentandioic acid is administered at 24 h to 48 h after radiation exposure.

In certain embodiments, a first dose of imidazolyl ethanamide pentandioic acid is administered at 6 h to 72 h after radiation exposure. In certain embodiments, a first dose of imidazolyl ethanamide pentandioic acid is administered at 8 h to 48 h after radiation exposure. In certain embodiments, a first dose of imidazolyl ethanamide pentandioic acid is administered at 12 h to 24 h after radiation exposure.

In certain embodiments, the radiation dose is between 0.2 Gy and 35 Gy of total body irradiation.

In certain embodiments, the radiation dose is between 0.2 Gy and 13.5 Gy of total body irradiation.

In certain embodiments, the radiation dose is between 0.2 Gy and 4.0 Gy of daily total body radiation.

In certain embodiments, the radiation dose is between 20 Gy and 80 Gy of focal radiation.

In certain embodiments, the radiation dose is between 1.8 Gy and 30 Gy of daily focal radiation. In certain embodiments, the radiation dose is between 1.8 Gy and 2.0 Gy of daily focal radiation.

In certain embodiments, the radiation dose is between 1.5 Gy and 30 Gy of daily focal radiation in a patient of age 2 to 17, particularly 2 to 16, more particularly 3 to 16 (pediatric radiation therapy). In certain embodiments, the radiation dose is between 1.5 and 1.8 Gy of daily focal radiation in a patient of age 2 to 17, particularly 2 to 16, more particularly 3 to 16 (pediatric radiation therapy).

In certain embodiments, the radiation is received as an acute lethal or near lethal dose sufficient to generate symptoms associated with Acute Radiation Syndrome (ARS). In certain embodiments, the radiation generates delayed effects of acute radiation exposure (DEARE), which includes myriads of chronic illnesses affecting multiple organ systems.

In certain embodiments, the radiation-induced damage is caused by radiation therapy, by radioisotope contamination (e.g. accidental leak of a nuclear reactor), by chronic low dose cosmic radiation or by the radiation of a nuclear weapon. In certain embodiments, the radiation-induced damage is caused by radiation therapy in cancer treatment.

In certain embodiments, the radiation therapy comprises X-ray, gamma or neutron radiation. In certain embodiments, the radiation therapy uses as its emitting source Co60, 137 Cs, iodine-131, lutetium-177, yttrium-90, radium-223, strontium-89, samarium (153Sm), or lexidronam.

In certain embodiments, imidazolyl ethanamide pentandioic acid is administered orally, intraperitoneally and intravenously, particularly orally.

In certain embodiments, imidazolyl ethanamide pentandioic acid is administered daily for at least three days. In certain embodiments, imidazolyl ethanamide pentandioic acid is administered daily for five to ten days.

In certain embodiments, imidazolyl ethanamide pentandioic acid is administered daily for at least three days after radiation exposure. In certain embodiments, imidazolyl ethanamide pentandioic acid is administered daily for five to ten days after radiation exposure.

In certain embodiments, the radiation-induced damage is caused by ionizing radiation. In certain embodiments, the radiation-induced damage is caused by photon radiation.

In certain embodiments, the treatment modality comprises external-beam radiation therapy, particularly the external-beam radiation therapy is selected from the group consisting of intensity-modulated radiation therapy (IMRT), image-guided radiation therapy (IGRT), tomotherapy, stereotactic radiosurgery, stereotactic body radiation therapy, photon beam, electron beam and proton or neutron therapy.

In certain embodiments, the radiation therapy comprises internal radiation therapy. In certain embodiments, the radiation therapy comprises brachytherapy. In certain embodiments, the radiation therapy comprises systemic radiation therapy. In certain embodiments, the radiation therapy comprises therapeutic accidental radiation overexposure (e.g. iatrogenic overdosing or handling accidents).

In certain embodiments, imidazolyl ethanamide pentandioic acid is administered in combination with G-CSF, GM-CSF, lipeg-G-CSF, peg-G-CSF or peg-GM-CSF. In certain embodiments, G-CSF, GM-CSF, lipeg-G-CSF, peg-G-CSF or peg-GM-CSF is administered at a dose of 2 μg/kg b.w./day to 30 μg/kg b.w./day. In certain embodiments, G-CSF, GM-CSF, lipeg-G-CSF, peg-G-CSF or peg-GM-CSF is administered at a dose of 2.8 μg/kg b.w./day to 10 μg/kg b.w./day.

A second aspect of the invention relates to a combination medicament for use in treatment or prevention of radiation-induced damage. The combination medicament comprises
imidazolyl ethanamide pentandioic acid and
G-CSF, GM-CSF, lipeg-G-CSF, peg-G-CSF or peg-GM-CSF.

Wherever alternatives for single separable features such as, for example, a dosage regimen or medical indication are laid out herein as “embodiments”, it is to be understood that such alternatives may be combined freely to form discrete embodiments of the invention disclosed herein. Thus, any of the alternative embodiments for a dosage regimen may be combined with any of the alternative embodiments of medical indication mentioned herein.

The invention is further illustrated by the following examples and figures, from which further embodiments and advantages can be drawn. These examples are meant to illustrate the invention but not to limit its scope.

### BRIEF DESCRIPTION OF THE FIGURES

FIG. 1 Kaplan-Meier survivor function in the untreated group, the vehicle group and in three groups with prophylactic treatment prior to a radiation dose of 5.8 Gy.

FIG. 2 Kaplan-Meier survivor function in the untreated group, the vehicle group and in two groups with therapeutic treatment after a radiation dose of 5.8 Gy.

FIG. 3 Kaplan-Meier survivor function in the untreated group, the vehicle group and in a single group with therapeutic treatment after a radiation dose of 6 Gy.

FIG. 4 (A): Percentage of animals in graded categories for posture (part of the ARS score) on Day 0, 3, 9, 15, 21, and 30 (AM) of groups 1 (U; 1), group 2 (VL (POx1); 2), group 3 (ML (IPx2); 3), group 4 (ML (POx2); 4), group 5 (ML (POx1); 5). (B): Percentage of animals in graded categories for posture (part of the ARS score) on Day 0, 3, 9, 15, 21, and 30 (AM) of groups 1 (U; 1), group 2 (VL (POx1); 2), group 6 (GL (SCx1); 6) and group 7 (M/GL (PO/SCx1); 7). (C): Percentage of animals in graded categories for posture (part of the ARS score) on Day 0, 3, 9, 15, 21, and 30 (AM) of group 1 (U; 1); group 8 (VH (POx1); 8) and group 9 (MH: (POx1); 9).

FIG. 5 (A): Percentage of animals in graded categories for coat (part of the ARS score) on Day 0, 3, 9, 15, 21, and 30 (AM) of groups 1 (U; 1), group 2 (VL (POx1); 2), group 3 (ML (IPx2); 3), group 4 (ML (POx2); 4), group 5 (ML (POx1); 5). (B): Percentage of animals in graded categories for coat (part of the ARS score) on Day 0, 3, 9, 15, 21, and 30 (AM) of groups 1 (U; 1), group 2 (VL (POx1); 2), group 6 (GL (SCx1); 6) and group 7 (M/GL (PO/SCx1); 7). (C): Percentage of animals in graded categories for coat (part of the ARS score) on Day 0, 3, 9, 15, 21, and 30 (AM) of group 1 (U; 1); group 8 (VH (POx1); 8) and group 9 (MH: (POx1); 9).

FIG. 6 (A): Percentage of animals in graded categories for behavior (part of the ARS score) on Day 0, 3, 9, 15, 21, and 30 (AM) of groups 1 (U; 1), group 2 (VL (POx1); 2), group 3 (ML (IPx2); 3), group 4 (ML (POx2); 4), group 5 (ML (POx1); 5). (B): Percentage of animals in graded categories for behavior (part of the ARS score) on Day 0, 3, 9, 15, 21, and 30 (AM) of groups 1 (U; 1), group 2 (VL (POx1); 2), group 6 (GL (SCx1); 6) and group 7 (M/GL (PO/SCx1); 7). (C): Percentage of animals in graded categories for behavior (part of the ARS score) on Day 0, 3, 9, 15, 21, and 30 (AM) of group 1 (U; 1); group 8 (VH (POx1); 8) and group 9 (MH: (POx1); 9).

FIG. 7 (A): Irradiation effect on body weight over time in Group 8 (VH (POx1); 8) (change of body weight over time relative to baseline weight). (B): Treatment effect on body weight over time in Group 9 (MH (POx1); 9) (change of body weight over time relative to control group 8 (VH (POx1); 8)) between day 3 and 30. (C): Body weight profiles measured over time from Day 0 to Day 30 of individual animals (blue) in untreated group (U; 1), vehicle group 8 (VH (POx1); 8) and Myelo001 treated group 9 (MH (POx1); 9). LOWESS smoothed mean weight in each treatment group is shown (red).

FIG. 8 White blood cell counts. (A): White blood cell counts on Day 0, 7, 14 and 30 of groups 1 (U; 1), group 2 (VL (POx1); 2) group 3 (ML (IPx2); 3), group 4 (ML (POx2); 4), group 5 (ML (POx1); 5) (box plots: median, 25%-75%, lower/upper adjacent values, outside values). (B): White blood cell counts on Day 0, 7, 14 and 30 of groups 1 (U; 1), group 2 (VL (POx1); 2), group 6 (GL (SCx1); 6) and group 7 (M/GL (PO/SCx1); 7) (box plots: median, 25%-75%, lower/upper adjacent values, outside values). (C): White blood cell counts on Day 0, 7, 14 and 30 of group 1 (U; 1), group 8 (VH (POx1); 8) and group 9 (MH: (POx1); 9) (box plots: median, 25%-75%, lower/upper adjacent values, outside values).

FIG. 9 Absolute neutrophil counts. (A): ANCs on Day 0, 7, 14 and 30 of groups 1 (U; 1), group 2 (VL (POx1); 2), group 3 (ML (IPx2); 3), group 4 (ML (POx2); 4), group 5 (ML (POx1); 5) (box plots: median, 25%-75%, lower/upper adjacent values, outside values). (B): ANC on Day 0, 7, 14 and 30 of groups 1 (U; 1), group 2 (VL (POx1); 2), group 6 (GL (SCx1); 6) and group 7 (M/GL (PO/SCx1); 7) (box plots: median, 25%-75%, lower/upper adjacent values, outside values). (C): ANC on Day 0, 7, 14 and 30 of group 1 (U; 1), group 8 (VH (POx1); 8) and group 9 (MH: (POx1); 9) (box plots: median, 25%-75%, lower/upper adjacent values, outside values).

FIG. 10 Absolute lymphocyte counts. (A): Absolute Lymphocyte Counts on Day 0, 7, 14 and 30 of groups 1 (U; 1), group 2 (VL (POx1); 2), group 3 (ML (IPx2); 3), group 4 (ML (POx2); 4), group 5 (ML (POx1); 5) (box plots: median, 25%-75%, lower/upper adjacent values, outside values). (B): Absolute Lymphocyte Counts on Day 0, 7, 14 and 30 of groups 1 (U; 1), group 2 (VL (POx1); 2), group 6 (GL (SCx1); 6) and group 7 (M/GL (PO/SCx1); 7) (box plots: median, 25%-75%, lower/upper adjacent values, outside values). (C): Absolute Lymphocyte Counts on Day 0, 7, 14 and 30 of group 1 (U; 1), group 8 (VH (POx1); 8) and group 9 (MH: (POx1); 9) (box plots: median, 25%-75%, lower/upper adjacent values, outside values).

FIG. 11 Absolute platelet counts. (A): Absolute Platelet Counts on Day 0, 7, 14 and 30 of groups 1 (U; 1), group 2 (VL (POx1); 2), group 3 (ML (IPx2); 3), group 4 (ML (POx2); 4), group 5 (ML (POx1); 5) (box plots: median, 25%-75%, lower/upper adjacent values, outside values). (B): Absolute Platelet Counts on Day 0, 7, 14 and 30 of groups 1 (U; 1), group 2 (VL (POx1); 2), group 6 (GL (SCx1); 6) and group 7 (M/GL (PO/SCx1); 7) (box plots: median, 25%-75%, lower/upper adjacent values, outside values). (C): Absolute Platelet Counts on Day 0, 7, 14 and 30 of group 1 (U; 1), group 8 (VH (POx1); 8) and group 9 (MH: (POx1); 9) (box plots: median, 25%-75%, lower/upper adjacent values, outside values).

FIG. 12 Hemoglobin. (A): Hemoglobin (g/dL) on Day 0, 7, 14 and 30 of groups 1 (U; 1), group 2 (VL (POx1); 2), group 3 (ML (IPx2); 3), group 4 (ML (POx2); 4), group 5 (ML (POx1); 5) (box plots: median, 25%-75%, lower/upper adjacent values, outside values). (B): Hemoglobin (g/dL) on Day 0, 7, 14 and 30 of group 1 (U; 1), group 2 (VL (POx1); 2), group 6 (GL (SCx1); 6) and group 7 (M/GL (PO/SCx1); 7) (box plots: median, 25%-75%, lower/upper adjacent values, outside values). (C): Hemoglobin (g/dL) on Day 0, 7, 14 and 30 of group 1 (U; 1); group 8 (VH (POx1); 8) and group 9 (MH: (POx1); 9) (box plots: median, 25%-75%, lower/upper adjacent values, outside values).

FIG. 13 Testes. (A): Percentage of severity of testes degeneration according to four categories (minimal, mild, moderate and marked) in group 2 (VL (POx1); 2), group 3 (ML (IPx2); 3), group 4 (ML (POx2); 4) and group 5 (ML (POx1); 5). (B): Percentage of severity of testes degeneration according to four categories (minimal, mild, moderate and marked) in group 2 (VL (POx1);2), 6 (GL (SCx1); 6) and 7 (M/GL (PO/SCx1); 7). (C): Percentage of severity of testes degeneration according to four categories (minimal, mild, moderate and marked) in group 8 (VH (POx1); 8 and 9 (MH (POx1); 9).

FIG. 14 Bone marrow cellularity. (A): Percentage of bone marrow cellularity decrease according to four categories (minimal, mild, moderate and marked) in group 2 (VL (POx1); 2), group 3 (ML (IPx2); 3), group 4 (ML (POx2); 4) and group 5 (ML (POx1); 5). (B): Percentage of bone marrow cellularity decrease according to four categories (minimal, mild, moderate and marked) in group 2 (VL (POx1);2), 6 (GL (SCx1); 6) and 7 (M/GL (PO/SCx1); 7). (C): Percentage of bone marrow cellularity decrease according to four categories (minimal, mild, moderate and marked) in group 8 (VH (POx1); 8 and 9 (MH (POx1); 9).

### EXAMPLES

### Materials and Methods

### Protocol and Study Execution

This study complied with the Protocol and SNBL USA (currently Altasciences) standard operating procedures (SOPs). Deviations and events that affected the quality or integrity of the data have additionally been described in applicable report sections.

The initial day of irradiation was designated as Day 0, with subsequent days consecutively numbered. Days on study prior to irradiation were consecutively numbered with the final day of acclimation designated as Day −1.

### Radiation and Dosimetry

Based on a previously reported study (Williams et al. 2010, Plett et al. 2012, Chua et al. 2014, Singh et al., 2015), the mouse model was developed to investigate LD25 and LD50. The whole-body irradiation and selected dose serve as a translational in vivo model mimicking possible radiation exposure to humans following a nuclear incident.

### Radiation

| | |
|---|---|
| Source/Model | X-Ray/Rad-Source RS-2000 |
| Target Dose Rate | 1.331 Gy/min |
| | Actual dose rates were measured pre |
| | and post-irradiation and ranged |
| | between 1.322 to 1.367 Gy/min. |
| Energy | 160 kV at 25 mA (on the floor of the |
| | RS-2000 chamber with circular RAD+) |
| Manufacturer | Rad Source Technologies, Inc. (Suwanee, GA) |
| Copper Filter Size | 0.3 mm Cu |
| Ion Chamber | RadCal 2086 Ion Chamber Dosimeter |
| Configuration | Shelf level on the floor of the chamber; the |
| | lead RAD+ shield in the center of |
| | the chamber floor; copper mesh on the floor; |
| | no turntable or additional circular |
| | copper mesh installed. |
| Dose Calculation | Dose (Gy) = Dose rate (Gy/min) * Time (min) |

### Substances

### Test Article

| | |
|---|---|
| Identification | Myelo001 (Imidazolyl ethanamide |
| | pentandioic acid; IEPA) |
| Supplier | Sponsor |
| Manufacturer | ERREGIERRE S.p.A. |
| | Via Francesco Baracca, 19 |
| | 24060 San Paolo d'Argon (BG) Italy |
| Lot/Batch | CAS N. 219694-63-0, Batch A170017 |
| Description | White or almost white, odorless |
| | crystalline powder |
| Purity | 99.6% |
| Retest Date | October 2020 |
| Storage Conditions | At 2 to 8° C. |

### Positive Control Article

| | |
|---|---|
| Identification | Neupogen ® (Filgrastim; Granulocyte |
| | colony-stimulating factor; G-CSF) |
| Supplier | SNBL USA |
| Manufacturer | Amgen |
| Lot/Batch | 1065526 |
| Description | Clear, colorless liquid |
| Concentration | 300 μg/mL |
| Expiration Date | 30 Apr. 2018 |
| Storage Conditions | At 2 to 8° C. |

### Vehicle (Negative Control)

| | |
|---|---|
| Identification | 0.5% aqueous |
| | hydroxypropylmethylcellulose (HPMC) |
| Manufacturer | Sigma |
| Lot/Batch | MKCB1715V |
| Description | White powder |
| Expiration Date | 16 Mar. 2023 |
| Storage Conditions | At 2 to 8° C. |

### Animals

SNBL USA, Ltd (hereafter, SNBL USA) is accredited by the Association for Assessment and Accreditation of Laboratory Animal Care (AAALAC), has an Animal Welfare Assurance issued by the Office of Laboratory Animal Welfare (OLAW), is registered with the United States Department of Agriculture (USDA), and has an Institutional Animal Care and Use Committee (IACUC) responsible for SNBL USA's compliance with applicable laws and regulations concerning the humane care and use of laboratory animals.

Animals (C57BL/6 mice) were supplied by Jackson Laboratories (Bar Harbor, Me. facility). Animals were maintained at SNBL USA (Everett, Wash. facility) as stock prior to study assignment and were screened for health by veterinary staff prior to use on study. 205 animals were assigned to treatment groups.

### Dosing

Oral application of Myelo001 would be conducive for nuclear or other radiation incidents and was chosen for this study. The dose of 50 mg/kg administered 3 days prior to irradiation was chosen. The human equivalent dose is estimated to be 4 mg/kg based on allometric calculations.

TABLE 1
Dosing

| | Number of | | Dose | | | | | |
|---|---|---|---|---|---|---|---|---|
| | Animals | Irradiation | Test/Control | | Treatment | Level | Conc. | Volumea |
| Group | (Male) | (Gy) | Article | Route | Timing | (mg/kg) | (mg/mL) | (mL/kg) |
| 1 | 5b + 5e | NA | NA | NA | NA | NA | NA | NA |
| 2 | 5c + 5d + 20e | LD | Vehicle | OG | Day −2, −1, | 0 | 0 | 10 |
| | | 25-5.8 | | | 0, 1, 2, 3 | | | |
| | | | | | (SID) | | | |
| 3 | 5c + 5d + 5e | | Myelo001 | IP | Day −3, −2, | 25 | 2.5 | 10 |
| 4 | 5c + 5d + 20e | | | | −1, 0 (6x) | 25 | 2.5 | 10 |
| 5 | 5c + 5d + 20e | | | OG | Day −2, −1, | 50 | 5 | 10 |
| | | | | | 0 (SID) | | | |
| 6 | 5c + 5d + 5e | | Neupogen | SC | Day 1, 2, 3 | 0.34 | 0.30 | 1.13 |
| 7 | 5c + 5d + 5e | | Myelo001/ | OG/SC | (SID) | 50/0.34 | 5/0.30 | 10/1.13 |
| | | | Neupogen | | | | | |
| 8 | 5c + 5d + 20e | LD | Vehicle | OG | | 0 | 0 | 10 |
| 9 | 5c + 5d + 20e | 50-6.0 | Myelo001 | | | 50 | 5 | 10 |

NA = not applicable;
Conc. = Concentration;
OG = orogastric;
IP = Intraperitoneal;
SC = Subcutaneous;
SID = Once a day;
LD = Lethal dose
x = Total Number of times doses administered (12 hours apart ±1 hour);
a = Total dose volume (mL) will be calculated based on the most recent body weight.
b = Necropsy on Day 0
c = Necropsy on Day 7
d = Necropsy on Day 14
e = Necropsy on Day 30

TABLE 2
Nomenclature and abbreviations of groups

| | Abbreviated | | |
|---|---|---|---|
| | Nomenclature | Nomenclature | |
| | used in graphs | used in graphs | |
| Group | and text | and text | Full text explanation |
| Group 1 | U; 1 | U; 1-untreated | Untreated Group 1 |
| Group 2 | VL; 2 | VL (POx1); 2 | Vehicle Low Irradiation |
| | | | (per os x1); Group 2 |
| Group 3 | ML; 3 | ML (IPx2); 3 | Myelo001 Low Irradiation |
| | | | (per ip x2); Group 3 |
| Group 4 | ML; 4 | ML (POx2); 4 | Myelo001 Low Irradiation |
| | | | (per os x2); Group 4 |
| Group 5 | ML; 5 | ML (POx1); 5 | Myelo001 Low Irradiation |
| | | | (per os x1); Group 5 |
| Group 6 | GL; 6 | GL (SCx1); 6 | G-CSF Low Irradiation |
| | | | (per sc x1); Group 6 |
| Group 7 | M/GL; 7 | M/GL | Myelo001/G-CSF |
| | | (PO/SCx1); 7 | Low Irradiation |
| | | | (per os/sc x1); Group 7 |
| Group 8 | VH; 8 | VH (POx1); 8 | Vehicle High Irradiation |
| | | | (per os x1); Group 8 |
| Group 9 | MH; 9 | MH (POx1); 9 | Myelo001 High Irradiation |
| | | | (per os x1); Group 9 |

### ARS Scoring

The primary outcome of this study was mortality and secondary outcomes were changes in hematology (peripheral and bone marrow). The endpoints were chosen to follow Animal Rule requirements that state that animal study endpoint be clearly related to the clinical benefit (generally, the enhancement of survival or prevention of major morbidity). The secondary endpoints were chosen to potentially contribute to an understanding of the disease or condition and a characterization of the treatment effect.

Scores on a scale of 1 to 4 (minimal, mild, moderate and severe) for posture, coat, and behavior were recorded twice daily per SNBL SOP, beginning on Day −3, except on the day of scheduled necropsy when scoring occurred only once. Observations were performed by 5 individuals during the course of the study.

After Day 0, the first ARS scoring began in the morning and the second ARS scoring began 4 to 6 hours following the completion of the morning ARS scoring. On the day of scheduled necropsy, ARS scoring occurred in the morning, prior to necropsy.

Based on the ARS Scoring SNBL SOP, if the sum of the three parameter scores totaled 8 or higher, the animal was considered moribund and was euthanized with an unscheduled necropsy performed according to the moribund animal SOP.

Cageside mortality checks were conducted per SOP, twice daily after Day −3. Morning and afternoon checks began 2 to 3 hours after the completion of the respective ARS scorings. Individual assessments were only documented for apparently moribund animals by re-scoring, or for found dead animals by removal.

Body weights were assessed twice during acclimation (including Day −3), once prior to irradiation (Day 0), and every 3 days thereafter. Terminal body weights were also collected, with the exception of Animal 4012.

### Statistical Analysis

Survival data were summarized descriptively and graphically using the Kaplan-Meier method. Further, death rates were tabulated by treatment. The equality of two or more survivor curves was tested with log rank tests. In addition, Cox-regression models were used to estimate hazard ratios, p-values and 95% confidence intervals (95% CI).

The focus of the survival analysis is irradiation related death between irradiation on Day 0 and Day 30. Thus, the scheduled necropsies on Day 7 and Day 14 or deaths unrelated to radiation (Animals 2041, 9012 and 9021) were considered as censored events.

Stata 14 (Stata Corp., 4905 Lakeway Drive, College Station, Tex. 77845, USA) was used for all additional calculations.

The longitudinal data were analyzed taking into account the mean weight over time in each group and the covariance among the repeated measures by response profile analysis (Fitzmaurice et al., Wiley 2004, p 103-140.). The analysis was specified as a regression model with unstructured covariance to account for the correlation among repeated body weights of the same mouse and indicator variables for treatment groups and time, where the vehicle treatment was used as the reference group and Day 0 was taken as a reference for time.

The response profile analyses provide the following regression coefficients estimated by the restricted maximum likelihood (REML) method:

intercept—mean body weight in the reference groups VL (POx1);2 and VH (POx1);8 at Day 0

treatment—difference between the mean body weight in the treatment groups and the vehicle groups at Day 0

time—body weight change from baseline in the reference groups

treatment×time—treatment effect at each day estimated by comparison of change from baseline in the treatment groups and control groups

For all regression coefficients, p-values and 95%-confidence intervals are provided. This method provides interpretable estimates, is valid if treatment groups differ at baseline, allows for arbitrary patterns in the mean body weight over time (no specific time trend e. g. linear curve assumed) and arbitrary patterns in the covariance. The analysis has certain robustness, as potential risks due to model misspecification are minimal.

### Example 1: Mortality

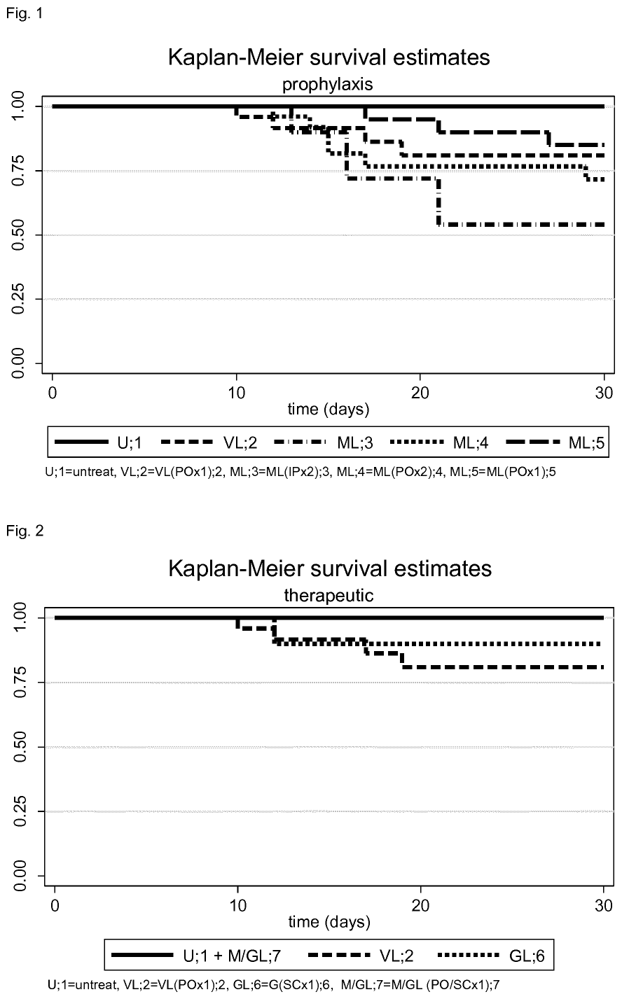

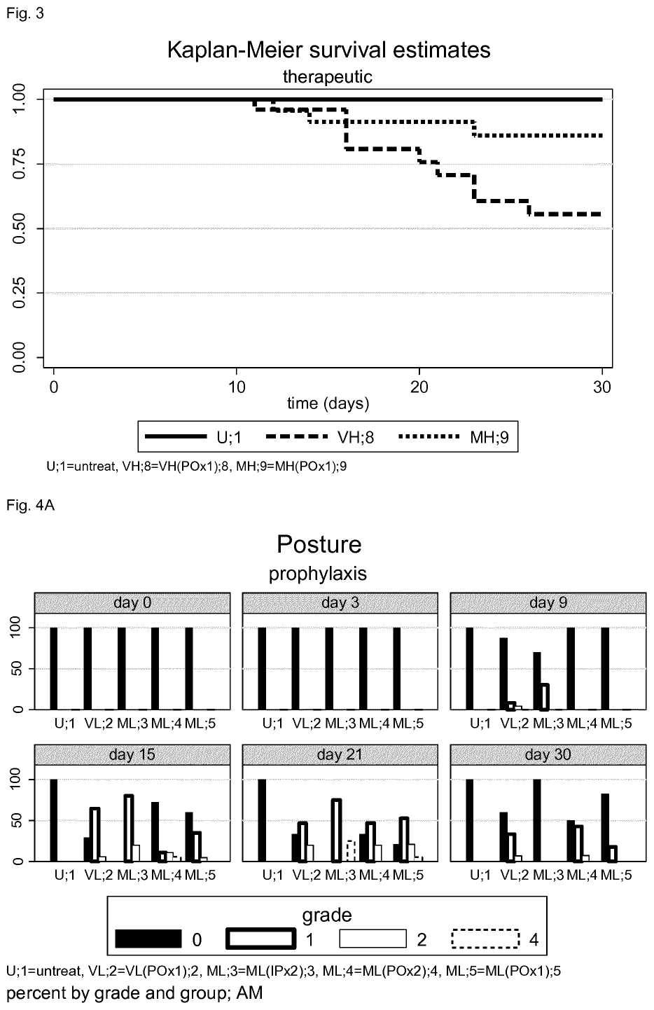

Summary of animal mortality data are included in table 3 to 4 and Kaplan-Meier plots are included in FIGS. 1 to 3. Animals 2041 and 9021 were euthanized on Day 3, and Animal 9012 was euthanized on Day 4. Based on historical data at SNBL, the onset of radiation-related symptoms generally occurs no sooner than Day 7. Therefore, irradiation effects were not considered to be the cause of moribund condition for these three animals. Additionally, gross pathology observations of fluid in the thoracic cavity for 2 of the 3 animals indicate that moribund condition was potentially related to dose administration. Therefore, these animals were excluded from mortality evaluations and all survival calculations.

An overview of the irradiation related death rates, the number of deaths divided by the time at risk is presented in table 3. The time to 10% death is shown in table 4. For the vehicle groups 2 (VL (POx1); 2) and 8 (VH (POx1); 8) rates of 0.65 and 1.48 per 100 days at a radiation dose of 5.8 and 6 Gy were observed. For the prophylactic groups rates between 0.44 and 1.30 per 100 days were found. For therapeutic treatment with G-CSF (group 6), a rate of 0.42 per 100 days was observed at the low radiation dose, whereas no death within 30 days occurred in the group 7 (M/GL (PO/SCx1); 7) with combination treatment. The rate in the group 9 (MH (POx1); 9) was lower than in the corresponding control group 8 (VH (POx1); 8) (0.48 vs. 1.48 per 100 days, respectively).

In the following, the survival is presented separately for prophylactic and therapeutic treatment. In the vehicle group, the time to 10% death is 17 days, for the prophylactic treatments estimates ranged from 13 to 21 days. The log rank test did not reveal a statistically significant difference between the survival curves of group 2 (VL (POx1); 2), group 3 (ML (IPx2); 3), group 4 (ML (POx2); 4) and group 5 (ML (POx1); 5) (p=0.337). Further analysis with Cox regressions, which controlled for the effect of treatment, did not suggest significant prophylactic effects (Tab. 5).

For the therapeutic treatment with G-CSF the time to 10% death is 12 days, in the group M/GL (PO/SCx1); 7 with combination treatment no death was observed until Day 30. The log rank test did not reveal significant differences between the survival curves of VL (POx1); 2, GL (SCx1); 6 and M/GL (PO/SCx1); 7 (p=0.469). An additional Cox regression, which controlled for the effect of treatment, did not suggest significant therapeutic effect for GL (SCx1); 6 (Tab. 6).

After the high radiation dose of 6 Gy, the time to 10% death is 16 days (95% CI 12 to 29) in the vehicle group and 23 days (95% CI 12 to n.a.) for the therapeutic treatment with Myelo001. Though the log rank test did not reach significance for a difference between the survival curves of VH (POx1); 8 and (p=0.066), the descriptive data suggests a positive effect in favor of MH (POx1); 9. A further analysis with a Cox regression controlling for the treatment effect, confirms this finding. The hazard ratio is 0.32 (p=0.085; 95% CI 0.09 to 1.17) (Tab. 7).

The dose reduction factor (DRF) computed as the ratio of the survival rate after 30 days in the vehicle and the corresponding prophylactic and therapeutic treatments adopts values up to 1.5. The maximum DRF of 1.5 was observed for the therapeutic treatment with Myelo001 at a high dose of 6 Gy (Tab. 8).

### Example 2: ARS Scores

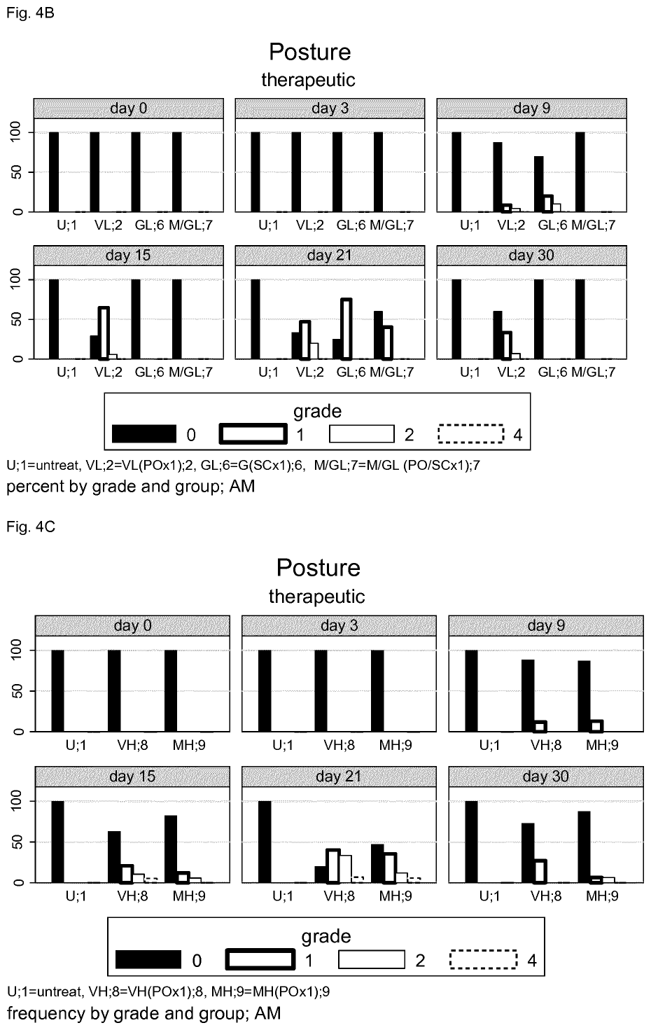

Significant differences in the score distribution of the posture of treated animals were observed on Day15. The most frequent grade (mode) observed in Groups 4 (ML (POx2); 4) and 5 ML (POx1); 5 was normal compared to control group 2 (VL (POx1); 2) (mild). Therapeutically treated Groups 6 (GL (SCx1); 6) and 7 (M/GL (PO/SCx1); 7) also showed the mode as normal in scoring of the posture, while control Group 2 (VL (POx1); 2) displayed predominantly (65%) as mild. On day 30, a trend for the increased frequency of normal posture score is observed in Group 9 (MH (POx1); 9) in comparison to control group 8 (VH (POx1); 8) (FIG. 4A-C, Tab. 10).

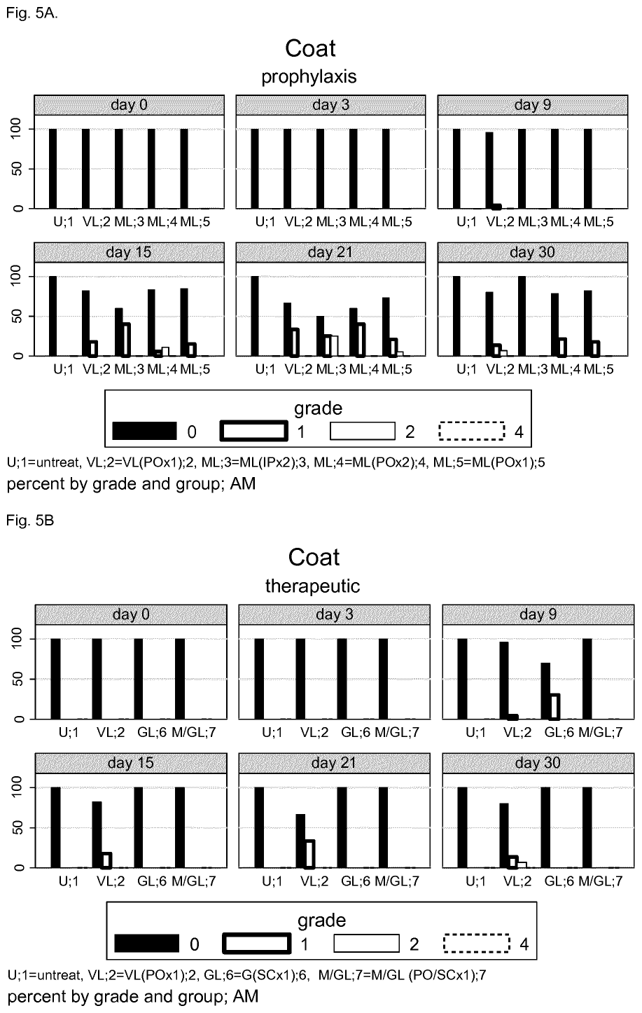

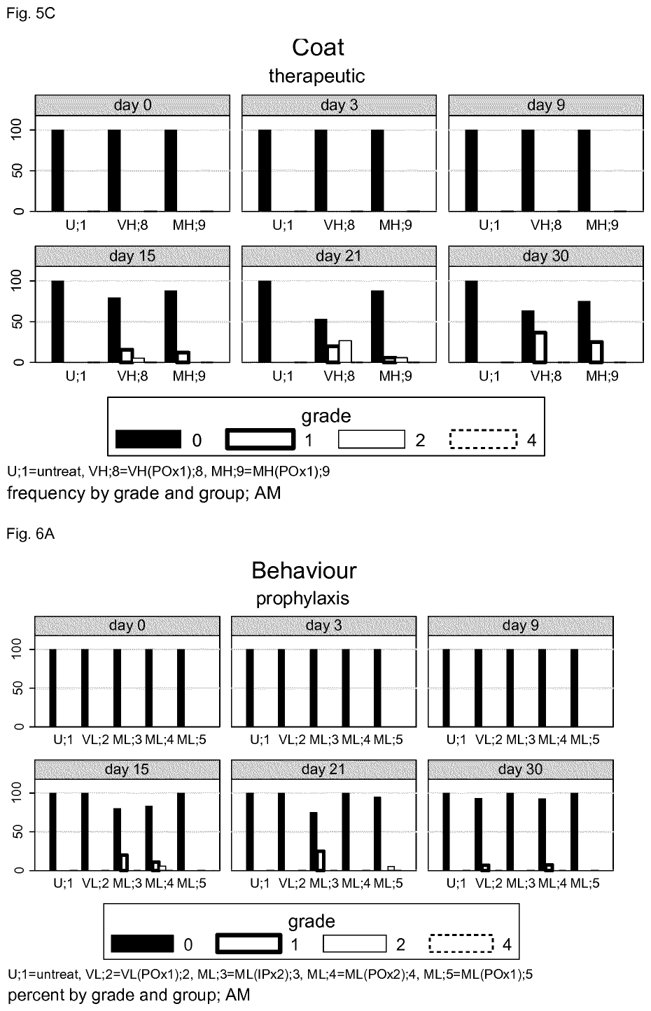

Overall, at Day 15, prophylactic treatment with Myelo001 (groups 4, 5) and therapeutic treatment with Myelo001 alone and Myelo001+Neupogen (groups 6, 7) resulted in the increased frequency of normal posture score in comparison with control group. Treatment with the low dose of Myelo001 (Group 3 (ML (IPx2); 3)) administered intraperitoneally did not result in such changes (FIG. 5A,B).

On Day 9, the ARS scoring for coat in Neupogen treated Group 6 (GL (SCx1); 6) showed increased percentage of mild grade, in contrast to vehicle treated Group 2 (2 (VL (POx1); 2) and Neupogen with Myelo001 treated Group 7 (M/GL (PO/SCx1); 7), where the coat was normal (FIG. 5B, Tab. 11).

In therapeutically treated groups irradiated with high dose 6 Gy, the coat grade of Myelo001 treated Group 9 (MH (POx1); 9) was mostly normal on Day 21, while in control Group 8 (VH (POx1); 8) mild (20%) and moderate (27%) grades can be observed (FIG. 5C, Tab. 11).

Overall, scoring for coat on Day 21 revealed most frequently the normal grade for Myelo001 treated Group 9 (MH: (POx1); 9) compared to mild and moderate mode in vehicle treated Group 8 (VH (POx1); 8).

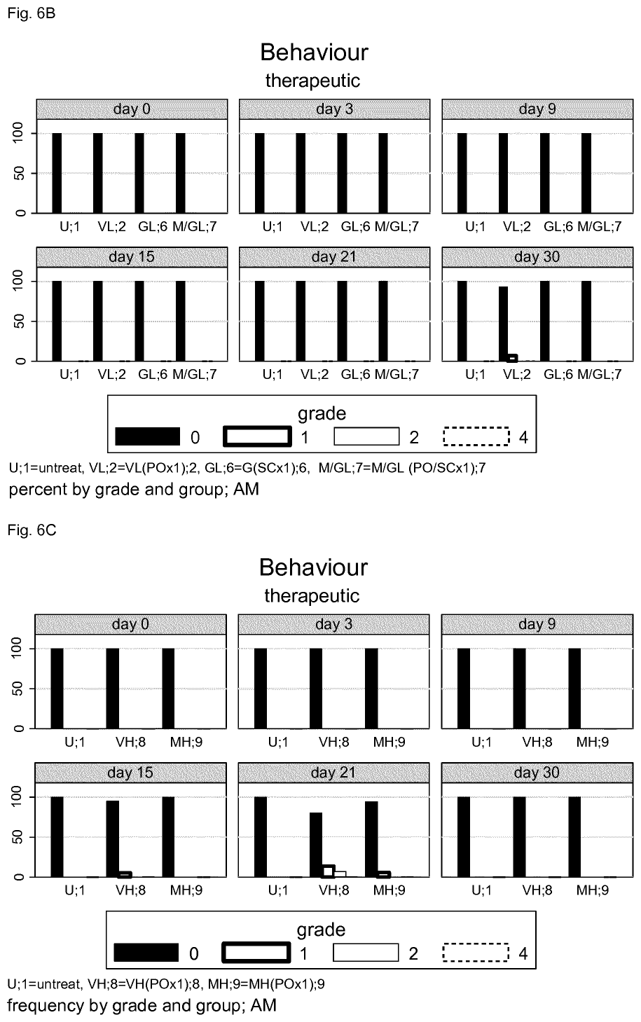

Radiation did not induce major changes in behavior scores of animals in all groups (Group 2 through 9) and statistical tests (Kruskal Wallis or Wilcoxon Rank-sum test) for showed no significant differences (FIG. 6A-C, Tab. 12).

Overall, there is a favorable though minor trend in ARS scores for posture and coat in favour (=lower scores) of the Myelo001 treated Group 9 compared to Group 8 (control). Therapeutically treated Group 6 (GL (SCx1); 6) and 7 (M/GL (PO/SCx1); 7) showed significant differences in the score distribution of the posture and coat.

### Example 3: Body Weight

Statistical analysis of body weight was performed on Days 0, 3, 9, 15, 21 and 30 (Tab. 13A-D). Data from Days 6, 12, 18, and 24 are not shown. The body weight changes are discussed separately for prophylactic and therapeutic treatment. The untreated group 1 (U; 1) gained weight over time.

### Prophylactic Treatment Result

The statistical results of the body weight for prophylactic treatment and subsequent irradiation with 5.8 Gy is summarized. The intercept suggests that the mean body weight in the vehicle group VL (POx1); 2 on baseline is 27.6 g (p<0.001; 95% CI: 26.9 to 28.2 g). The mean body weight in this control group decreases significantly from day 3 to day 30. The maximum decrease from baseline weight in the vehicle group is −3.7 g p<0.001; (−5.1 to −2.2 g) on day 21 (time). There is no evidence for different mean weights in the groups treated prophylactically with Melo001 relative to the vehicle group(treatment). The prophylactic treatment with Myelo001 tends to compensate the weight loss in the due to irradiation to a minor extent (treatment×time).

Small protective effect on Day 3 was shown for prophylactic groups 3 (ML (IPx2); 3), 4 (ML (POx2); 4) and 5 (ML (POx1); 5). For example, the mean body weight in the vehicle group is −1.9 g p<0.001; (−1.7 to −2.1 g) on day 3. The mean body weight in the ML (POx2); 4 group exceeds the vehicle group weight by 0.7 g (p<0.001; 95% CI: 0.4 to 1.0 g).

### Therapeutic Treatment Results

Therapeutic treatment with G-CSF (Group 6, (GL (SCx1); 6)) and combination treatment is summarized. The mean body weight of the vehicle group on day 0 is 27.6 g (p<0.001; 95% CI: 27.0 to 28.1 g). After irradiation at a level of 5.8 Gy the mean weight in this control group decreases significantly from baseline between Day 3 and Day 30 (time). The maximum weight loss is −4.3 g (p<0.001; 95% CI: −5.6 to −3.0 g). On day 0 there is no significant weight difference between the vehicle group VL (POx1); 2 and the GL (SCx1); 6 and M/GL (PO/SCx1); 7, respectively (treatment). At later time points the treatment tends to compensate the weight loss observed in the vehicle group. Therapeutic treatment with G-CSF (group 6, GL (SCx1); 6) resulted in a protective effect of 3.3 g (p=0.014; 95% CI: 0.7 to 5.9 g) on body weight on Day 21 relative to vehicle (Group 2, VL (POx1)). Similarly, treatment with both, Myelo001 and G-CSF, in Group 7 (M/GL (PO/SCx1); 7) led to an increase of 2.8 g (p=0.028, 95% CI: 0.3 to 5.3 g) body weight at Day 21 relative to vehicle (Group 2, VL (POx1)).

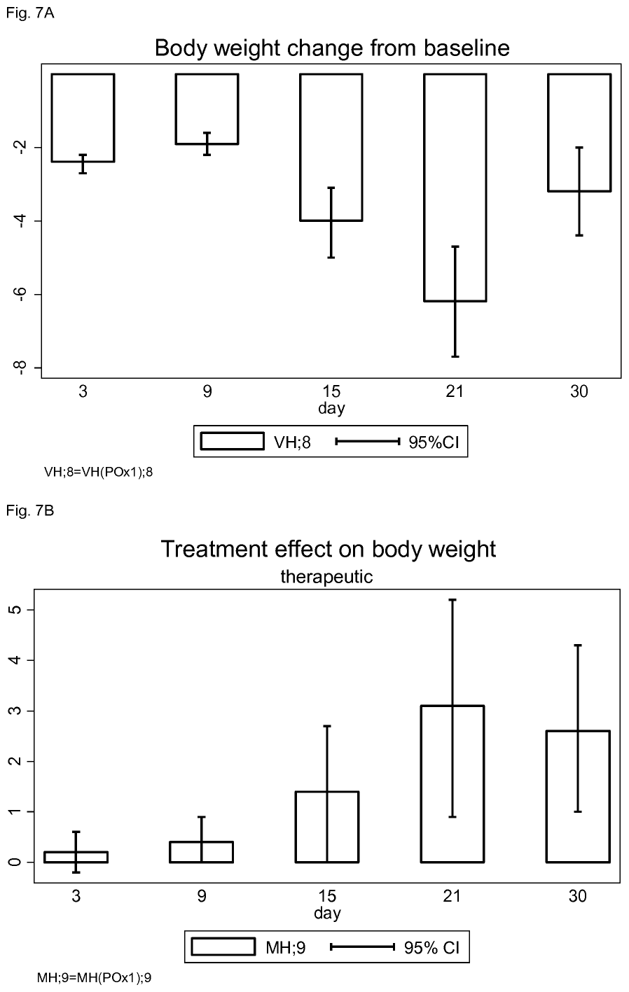

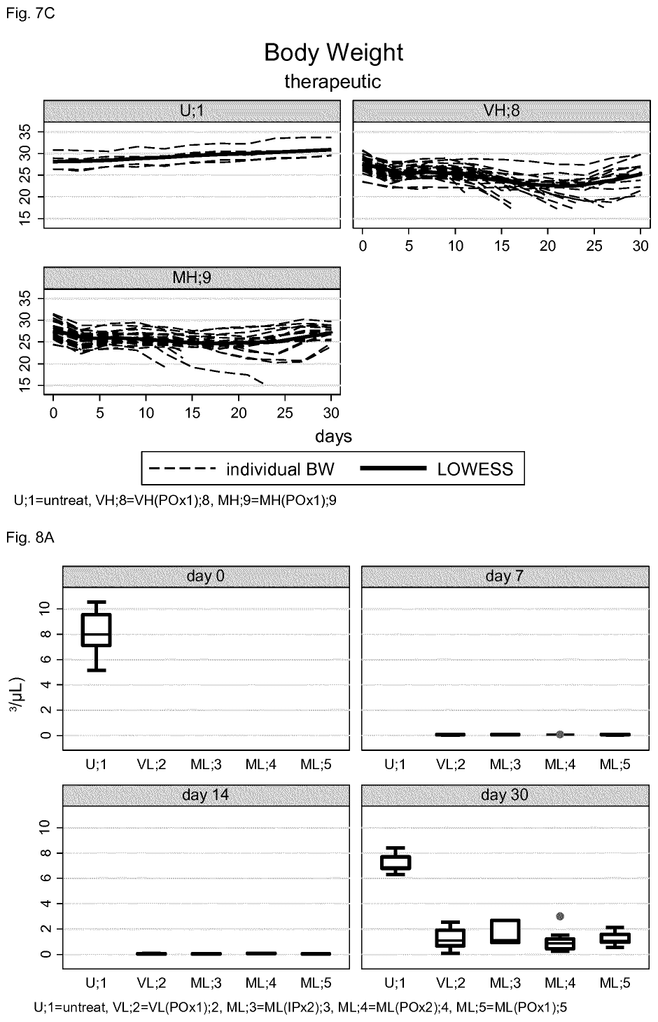

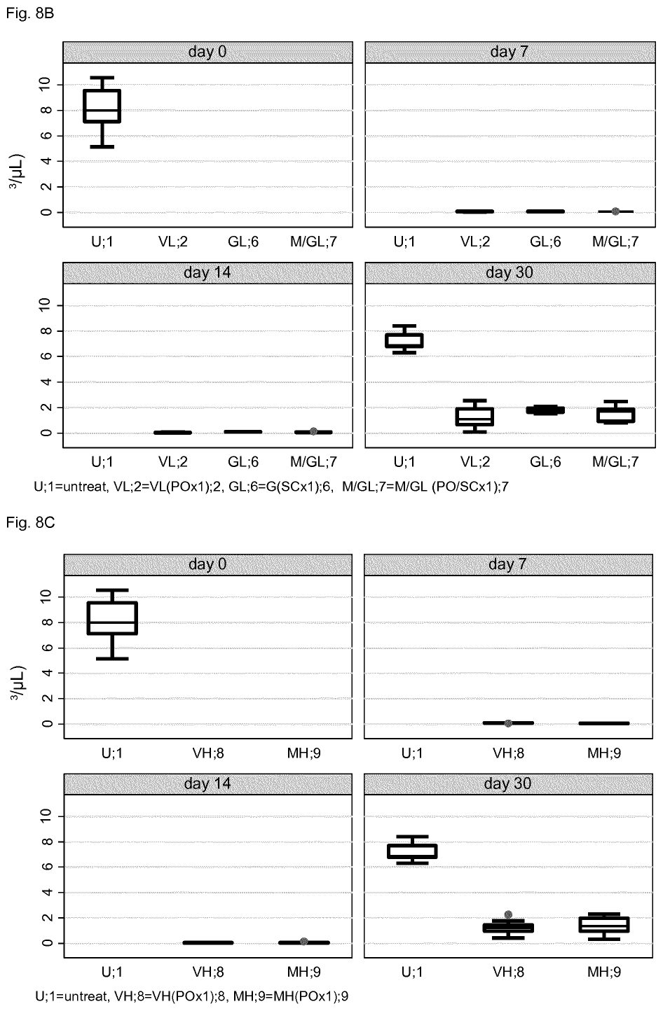

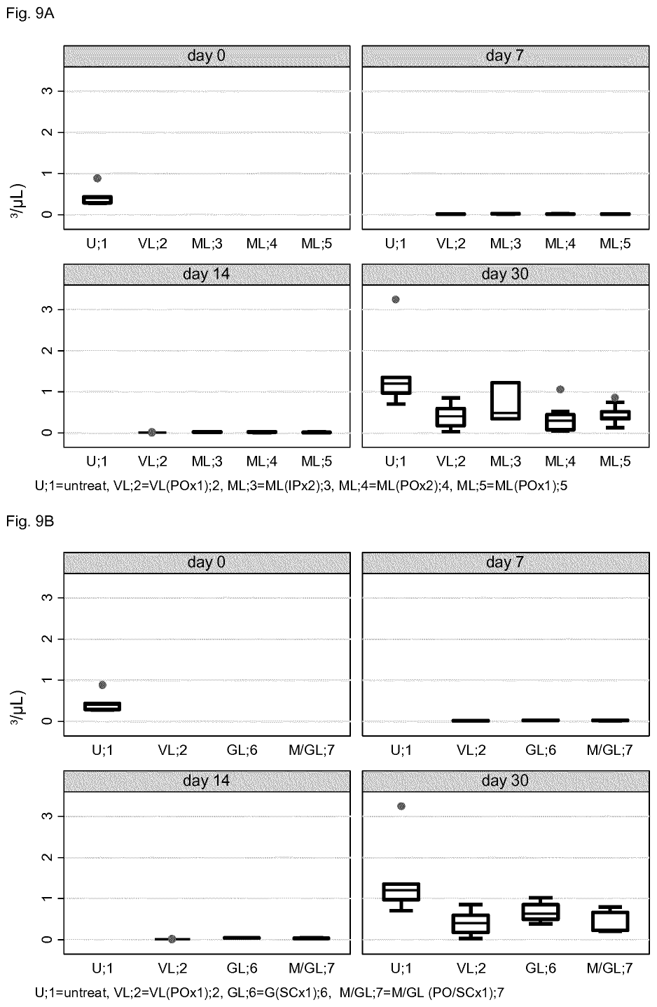

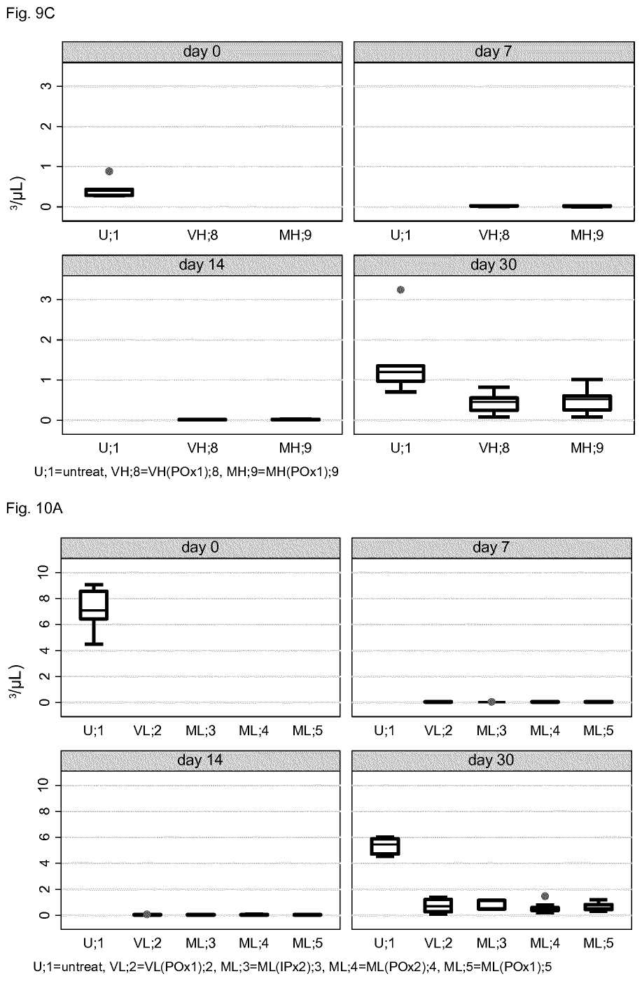

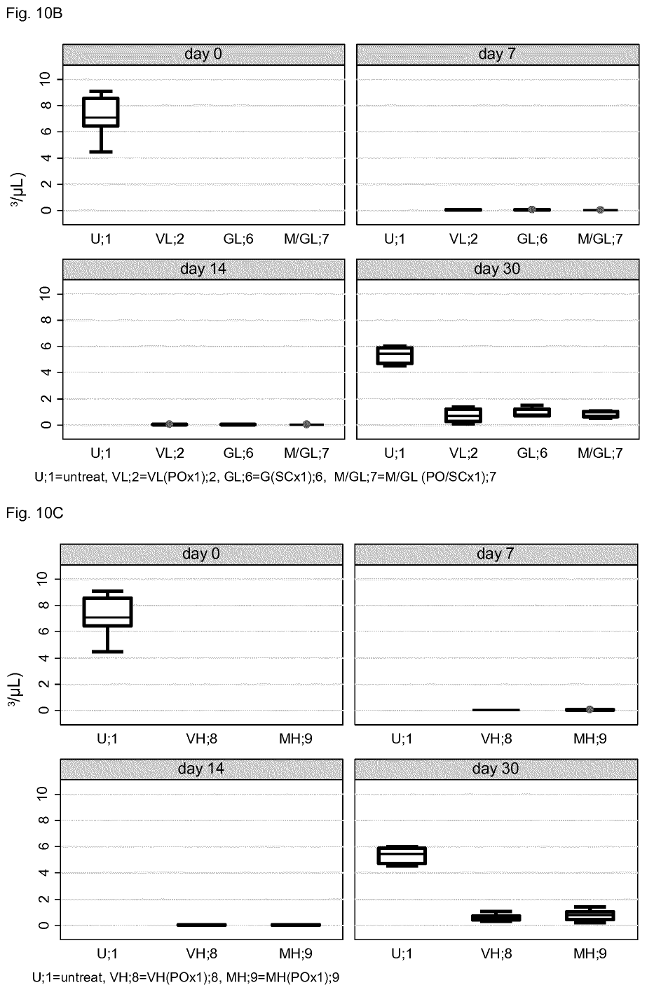

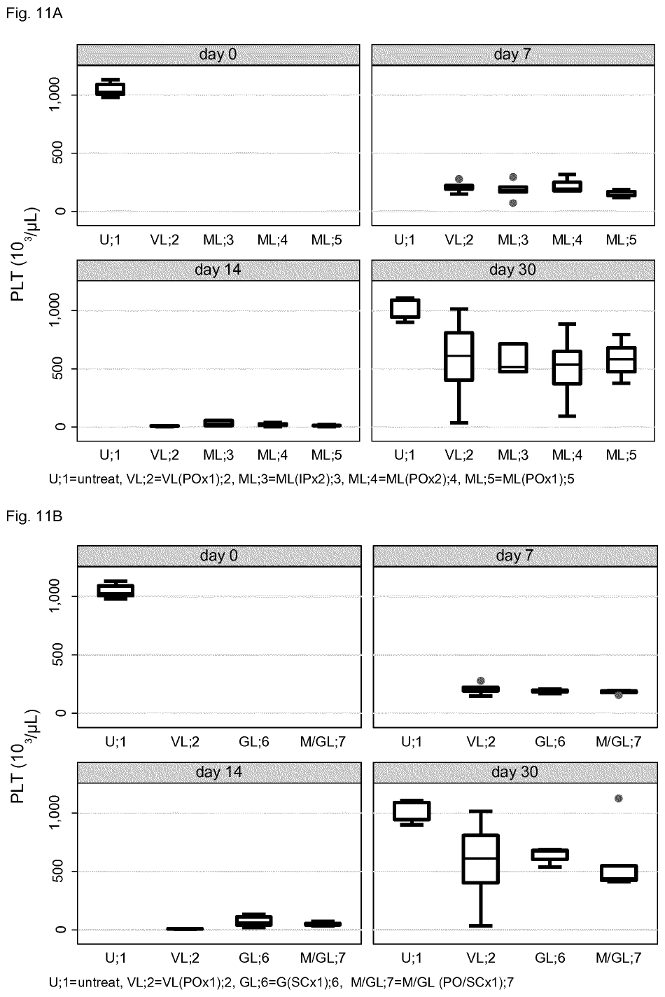

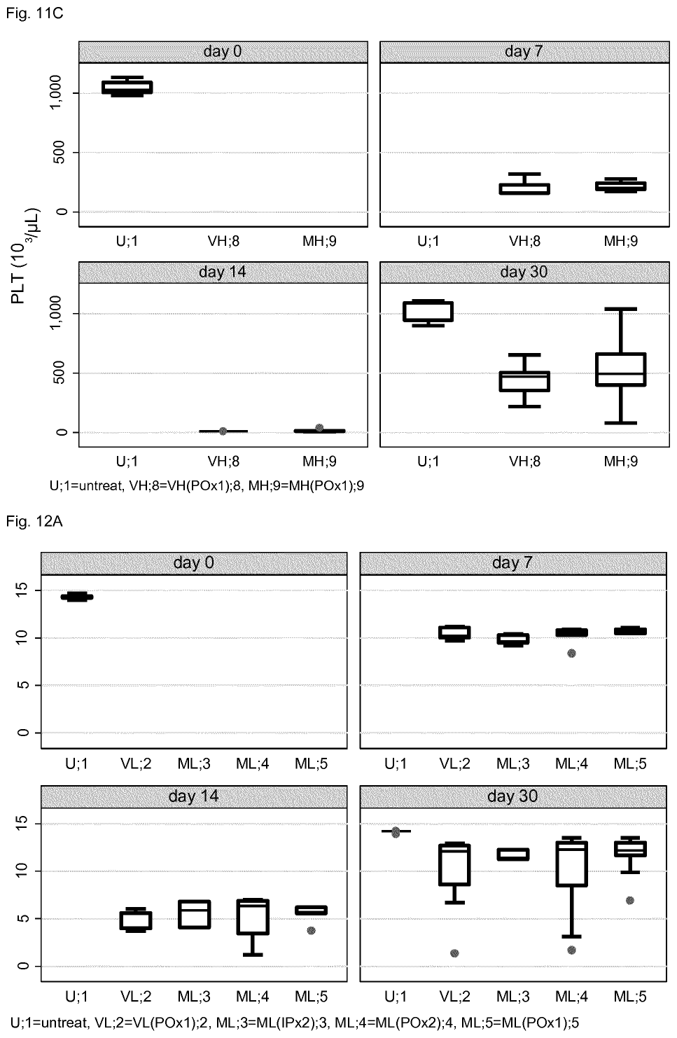

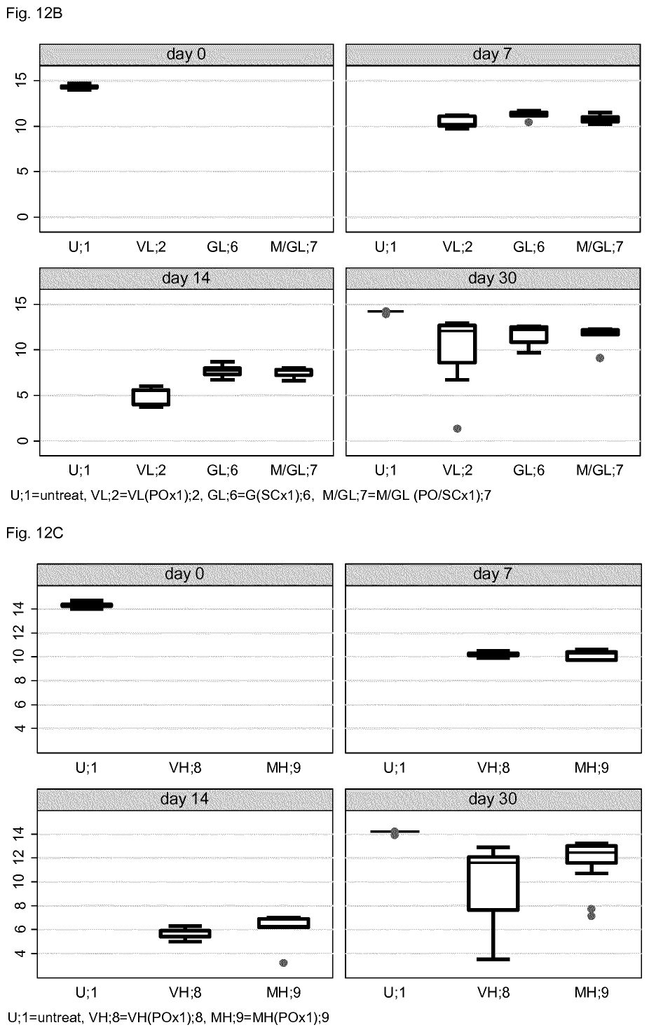

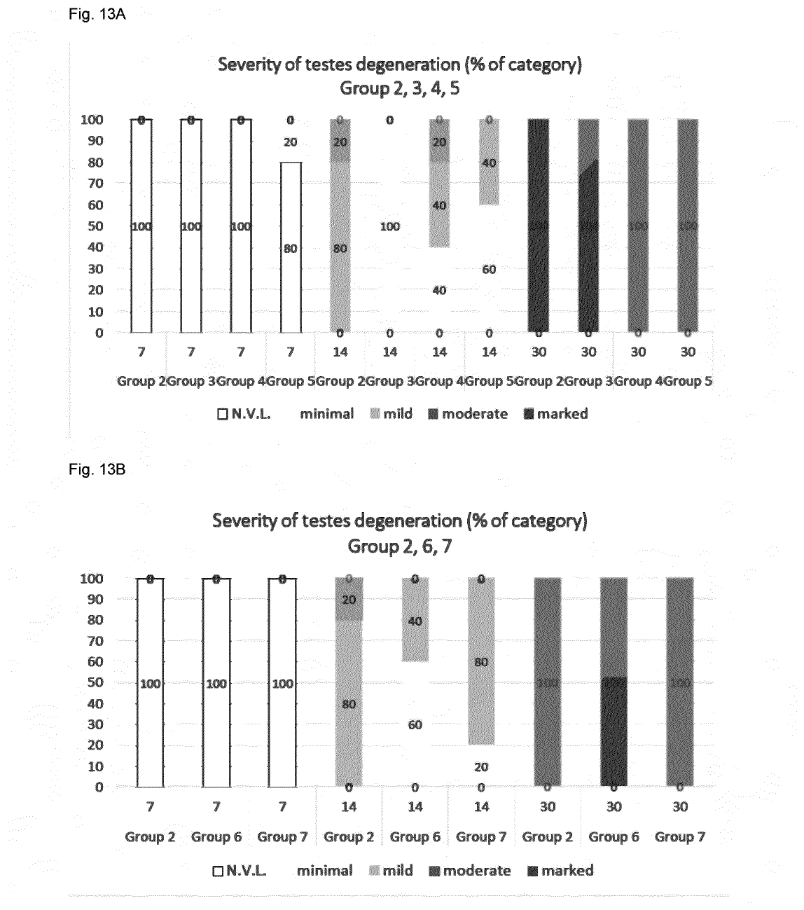

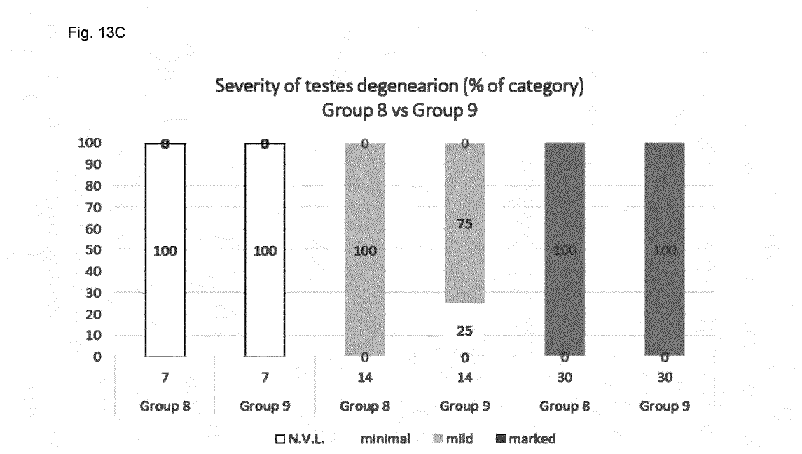

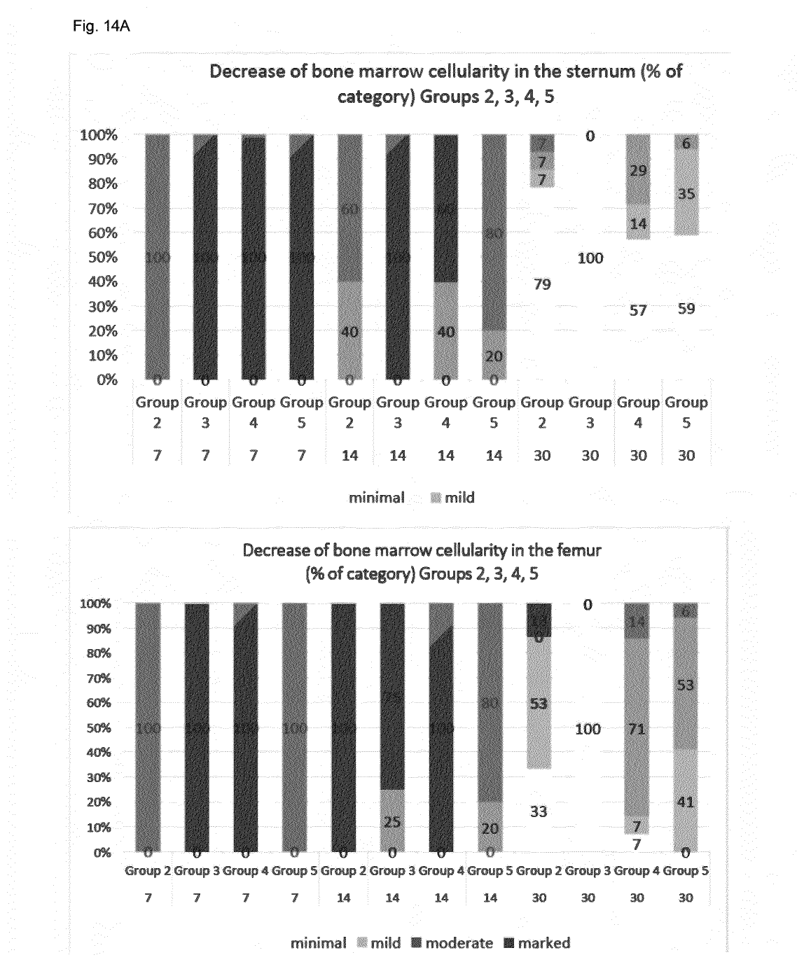

The body weight analysis of therapeutically treated mice after high dose irradiation is summarized. The intercept indicates that the mean body weight in the vehicle group VH (POx1); 8 on Day 0 is 27.7 g (p<0.001; 95% CL: 27.0 to 28.3 g). There is a significant decrease of body weight from baseline weight in the vehicle group from Day 3 to Day 30 as indicated by time. The maximum weight loss is −6.2 g (p<0.001; 95% CI: −7.7 to −4.7 g). This decrease after 6 Gy irradiation dose is more severe compared to 5.8 Gy irradiation. For baseline Day 0 no difference in mean body weight between vehicle and Group VH (POx1); 8 has been observed (treatment). Analysis of body weight changes over time in Group 9 (MH (POx1); 9) relative to control for Group 8 (VH (POx1); 8) (treatment×time) indicated a significant compensation of weight loss upon therapeutic treatment with Myelo001 relative to vehicle Group 8 (VH (POx1); 8) (FIG. 7A-C). This amounts up to 3.1 g (p=0.006; 95% CI: 0.9 to 5.2 g).

In summary, prophylactic treatment with Myelo001 resulted in a small protective effect on Day 3. Upon lower dose radiation, therapeutic treatment with Myelo001 and Myelo001 +Neupogen resulted in the increase of body weight on Day 21 relative to the vehicle Group 2 (VH (POx1); 2) on the same day. The highest protective effect on body weight of Myelo001 was observed in the therapeutic regimen under higher dose irradiation showing a positive effect on Days 15, 21, and 30 of Group 9 (MH (POx1); 9) relative to control Group 8 (VH (POx1); 8).

### Example 4: Hematology and Pathology

Peripheral Hematology (White Blood Cells, neutrophis, lymphocytes and platelets) was severely suppressed and Hemoglobin moderately suppressed in all groups (vs. the untreated/non-radiated group (U; 1)) on Day 7 and 14. White blood cells, lymphocytes and platelets remained largely suppressed at the last time point (Day 30), whereas neutrophils and hemoglobin returned close to normal values. Overall, no pronounced differences were observed upon prophylactic and therapeutic treatment with Myelo001 or the positive control G-CSF. The number of white blood cells, neutrophils or lymphocytes were comparable between radiated groups. On Day 14, hemoglobin and hematocrit, red blood cells, were increased in groups 6 (GL (SCx1); 6) and 7 (M/GL (PO/SCx1); 7) compared to control group 2 (VL (POx1); 2). Similarly, there was a trend on Day 14 and Day 30 for hemoglobin, red blood cells, hematocrit in favor of group 9 (MH: (POx1); 9) compared to group 8 (VH (POx1); 8) (FIG. 8A-C—12A-C).

On day 30, a trend in the increase of the median WBC and neutrophil count was observed in Group 6 (GL (SCx1; 6) and Group 7 (M/GL (PO/SCx1); 7) compared to control Group 2 (VH (POx1); 2). No significant group differences in the number of neutrophils could be identified including in the positive control (G-CSF). This may be due to the selected time points of 7 and 14 days with global suppression and missing measurement in the period of partial recovery as well as stopping at day 30.

There were no test article-related changes noted in the unscheduled mortality gross pathology data. Typical acute radiation syndrome findings (red discoloration in multiple organs, mainly in the brain and testes) were observed in these animals.

Lesions typical of acute radiation syndrome, namely red discoloration of organs, which were most prominent in the brain and testes, were observed in all groups during the Day 14 necropsy except Group 6 (5.8 Gy, Neupogen, subcutaneous, 0.34 mg/kg, administered on Days 1, 2 and 3), which had no red discoloration in the brain, testes, but cysts in spleen were observed in Group 6, which are not typical of acute radiation syndrome.

No other test article-related changes were noted in the Day 7 or 30 necropsy gross pathology data.

There were no test article-related changes were noted in the organ weights data.

Microscopic findings of hemorrhage in the testes, myeloid hyperplasia of the bone marrow, and megakaryocyte hyperplasia in the bone marrow were observed sporadically, including in vehicle-treated animals and could not be associated with the test article.

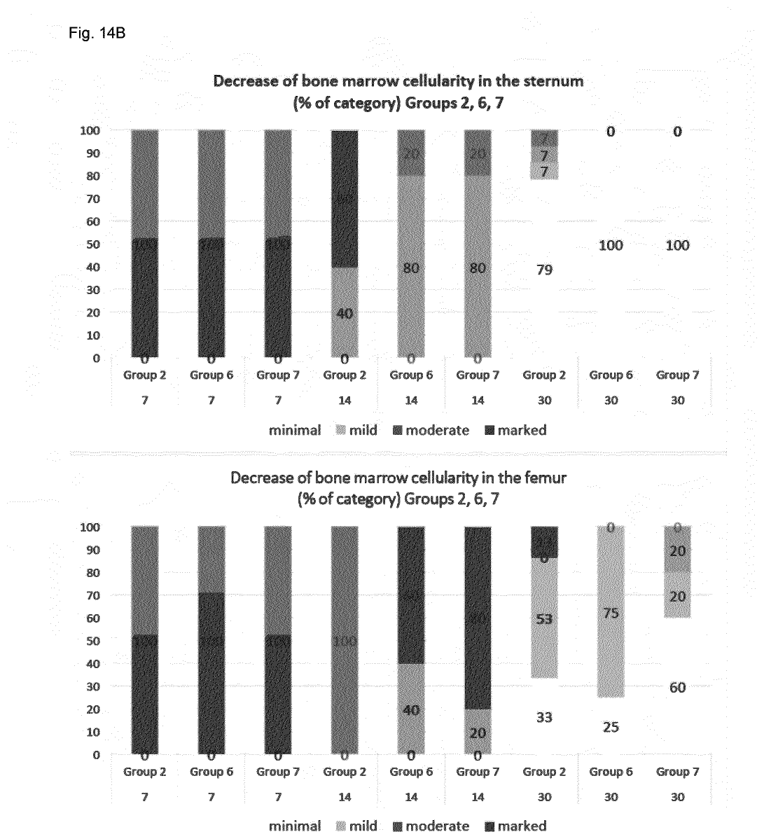

The severity of testes degradation was reduced in Group 3 (ML (IPx2); 3), Group 4 (ML (POx2); 4), Group 5 (ML (POx1); 5) compared to Group 2 (VL (POx1); 2) on Day 14 (p=0.037) (FIG. 13A, Tab. 14). On Day 30, the severity was marked in all groups. Animals in Group 6 (GL (SCx1); 6) and group 7 (M/GL (PO/SCx1); 7) showed higher proportions of testes in the minimal category but those changes were insignificant (p=0.078) (FIG. 13B). Similarly, animals treated with Myelo001 in Group 9 (MH (POx1);9) showed (n.s.) higher proportion of testes in minimal category (p=0.264) (FIG. 13C). No test article-related changes were noted in the scheduled Day 7 necropsy histopathology data. Decreased cellularity of the bone marrow in both sternum and femur was marked (Grade 4) in all animals. The testes were normal (no visible lesions).

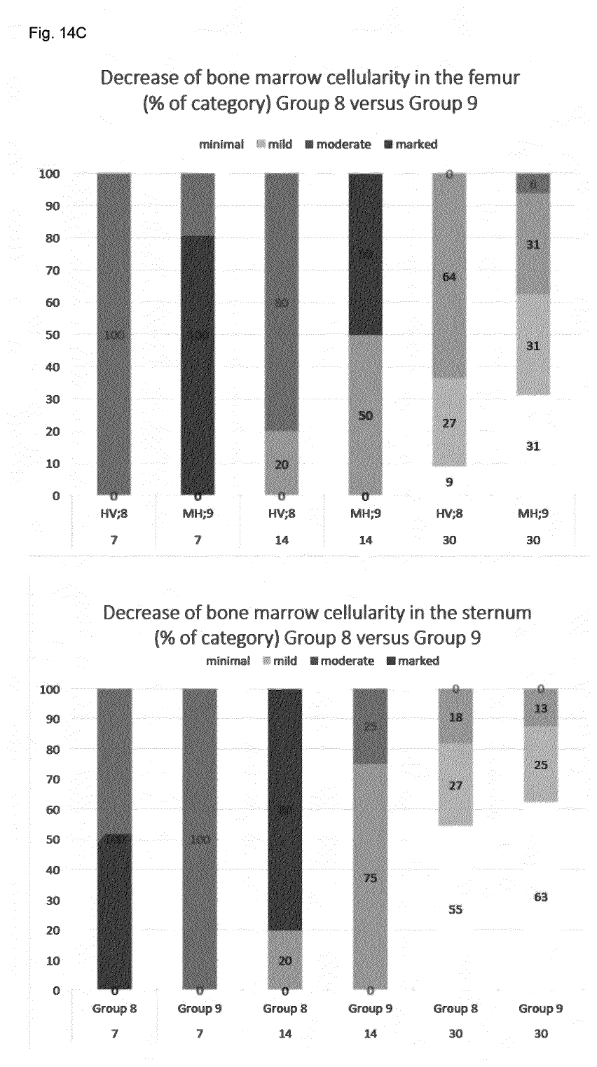

Bone marrow cellularity decrease was in the “marked” category in femur and sternum in 100% of radiated animals on Day 7 and recovered on Day 14 to moderate to marked. On Day 30, no animals other than Group 2 (VL (POx1); 2) had marked decrease in the sternum and the majority of animals in Group 2 (VL (POx1); 2) were in the minimal category. In the femur, moderate or marked decrease of cellularity was still present in most groups but recovery of cellularity was observed in all groups (FIG. 14A-C).

Group 2 (VL (POx1); 2), Group 4 (ML (POx2); 4) and Group 5 (ML (POx1); 5) did not show any significant changes in the decrease of bone marrow cellularity. For all animals in Group 3 (ML (IPx2); 3) bone marrow cellularity was in minimal category on Day 30 and the difference for femur bone marrow grades was significant (p=0.001) (FIG. 14A).

On Day 14, the control Group 2 (VL (POx1);2) showed (n.s.) higher proportion of animals in the marked category in comparison with Group 6 (GL (SCx1); 6) and Group 7 (M/GL (PO/SCx1); 7). On Day 30, all treated animals in Group 6 (GL (SCx1); 6) and Group 7 (M/GL (PO/SCx1); 7) showed minimal category in sternum and no marked category in femur bone marrow, but those changes were insignificant (FIG. 14B).

The control Group 8 (VH (POx1); 8) showed statistically non-significant higher proportions of animals in the moderate and marked category on Day 14 and 30 compared to Group 9 (MH (POx1); 9) (FIG. 14C, Tab. 15).

In summary, for the scheduled Day 14 and 30 necropsies, lower severity was noted of decreased cellularity in the bone marrow of both sternum and femur in Groups 6 (GL (SCx1); 6) and Group 7 (M/GL (PO/SCx1); 7) compared to vehicle-treated controls in Group 2 (VL (POx1);2). In the higher radiation dose groups, Group 9 (MH (POx1); 9) had lower severity of decreased cellularity of the bone marrow of both sternum and femur compared to vehicle-treated control in Group 8 (VH (POx1); 8)).

### CONCLUSIONS

Dose-dependent radiation effects were apparent in the primary endpoint of mortality and in most assessed secondary endpoint parameters, including body weight, clinical signs, hematology, and histopathology.

Hematologic assessment had limitations due to selected time points of 7 and 14 days that showed severe myelosuppression across all groups, with no further measurements scheduled until the end of the study on day 30, when recovery was still incomplete.

Prophylactic treatment after 5.8 Gy irradiation (LD25/30) did not result in a significant difference between the survival curves compared to control group 2 (VH (POx1; 2). However, oral administration of Myelo001 at the dose 50 mg/kg (Group 5 (ML (POx1); 5)) resulted in a higher dose reduction factor (DRF=1.1) when compared to oral and intraperitoneal administration of two doses of 25 mg/kg (Group 4 (ML (POx2); 4) and Group 3 (ML (IPx2); 3) (DRF=0.9 and 0.7, respectively). Additionally, in the vehicle group, the time to 10% death was 17 days, whereas in Group 5 (ML (POx1); 5) it was 21.

Prophylactic intraperitoneal treatment with Myelo001 (Group 3 (ML (IPx2); 3)) led to a lower decrease of the bone marrow cellularity on day 30 and decreased degeneration of the testes on Day 14 compared to vehicle control. No clear impact on the peripheral hematology and ARS scores was observed. Prophylactic oral administration of Myelo001 in Group 4 (ML (POx2); 4) and Group 5 (ML (POx1); 5) resulted in a decrease of testes degeneration on Day 14 and notable differences in the score distribution of the posture on Days 9 and 15. The most frequent grade (mode) of posture observed in both groups was normal compared to control group 2 (VL (POx1); 2) which was more frequently classified as mild. Bone marrow cellularity and hematology were not considerably altered by the treatment. All prophylactic treated groups (groups 3, 4, 5) showed a small protective effect on the body weight loss on day 3.

After the therapeutic treatment with the positive control G-CSF (Group 6 (GL (SCx1); 6)) only a minor difference in survival was observed compared to negative control (VL (POx1); 2), whereas under combination treatment (Group 7 ((M/GL (PO/SCx1)) no death occurred within 30 days. Mildly increased DRF was observed after administration of Myelo001 and G-CSF together (M/GL (PO/SCx1); 7) compared to G-CSF alone (GL (SCx1); 6) (DRF=1.2 vs 1.1, respectively).

Test article-related changes consisted of lower decrease in cellularity of the bone marrow in Groups 6 (GL (SCx1); 6) and 7 (M/GL (PO/SCx1); 7). This trend was observed in both femur and sternum at Days 14 and 30, with greater recovery at Day 30.

Irradiation resulted in pancytopenia in vehicle treated animals on Days 7 and 14. The decreases in the mean values for erythrocyte count (RBC mature cells and immature reticulocytes), hemoglobin (HGB), and hematocrit (HCT) on Day 14 were less pronounced in animals receiving G-CSF alone (Group 6 (GL (SCx1; 6)) or G-CSF in combination with Myelo001 (Group 7 (M/GL (PO/SCx1); 7). No clear group differences in the number of WBC and neutrophils could be identified including in the positive control (G-CSF), although a trend for the increase of the mean numbers of these cell lineages was noted in both groups. Overall, a positive trend of Myelo001 for the mitigation of hematopoietic acute radiation syndrome (H-ARS) was observed.

Severity of the testes degeneration was highest in vehicle control groups at Day 14. This may indicate some protective effect in treatment groups at this time point; however; the end result was the same at Day 30 for all animals, which was marked degeneration of the testes. Group 6 (GL (SCx1); 6) at Day 14 was the only group that did not have gross lesions in testes typical of acute radiation syndrome, but the significance of this is not known, especially as Group 7 (M/GL (PO/SCx1); 7) received similar treatment with the addition of another test article.

On day 21, both groups showed significant treatment effect on the increase of the body weight compared to control vehicle group.

For the therapeutic treatment after 6.0 Gy radiation (LD50/30), the survival in Group 9 (MH (POx1); 9) was substantially higher than in the corresponding control Group 8 (VH (POx1); 8) (86% vs. 56%, respectively). Group 9 (MH (POx1); 9) had the highest dose reduction factor of 1.5 of all treatment groups.

The increase in survival was supported by several positive trends in secondary parameters including bone marrow cellularity, testes degeneration and ARS score for coat and posture. Additionally, the highest protective effect on body weight of Myelo001 was observed in the therapeutic regimen under higher dose irradiation showing a significant effect on Days 15, 21 and 30 of Group 9 (MH (POx1); 9) relative to control Group 8 (VH (POx1); 8).

No clear changes were observed regarding WBC count. However, the decreases in the mean values for erythrocyte count (RBC mature cells and immature reticulocytes), hemoglobin (HGB), and hematocrit (HCT) on Days 14 and 30 was less pronounced after therapeutic oral administration of Myelo001 (Group 9 (MH (POx1); 9)). Overall, a positive trend of Myelo001 for the mitigation of hematopoietic acute radiation syndrome (H-ARS) was observed.

In summary, particularly for the therapeutic treatment after 6.0 Gy radiation (LD50/30), the survival in Group 9 (MH (POx1); 9) was substantially higher than in the corresponding control group 8 (VH (POx1); 8). This survival finding was supported by several positive trends in secondary parameters including bone marrow cellularity, ARS score and body weight.

TABLE 3
Irradiation related death rates

| | | Time at Risk | Rate (per | |
|---|---|---|---|---|
| Group | Dead | (Days) | 100 Days) | 95% Cl |
| untreated | 0 | 150 | 0.00 | n. a. |
| VL (POx1); 2 | 4 | 616 | 0.65 | 0.24 to 1.73 |
| ML (IPx2); 3 | 3 | 231 | 1.30 | 0.42 to 4.03 |
| ML (POx2); 4 | 6 | 627 | 0.96 | 0.43 to 2.13 |
| ML (POx1); 5 | 3 | 680 | 0.44 | 0.14 to 1.37 |
| GL (SCx1); 6 | 1 | 237 | 0.42 | 0.06 to 3.00 |
| M/GL | 0 | 255 | 0.00 | n. a. |
| (PO/SCx1); 7 | | | | |
| VH (POx1); 8 | 9 | 607 | 1.48 | 0.77 to 2.85 |
| MH (POx1); 9 | 3 | 611 | 0.48 | 0.15 to 1.48 |

TABLE 4
Time to 10% death

| Group | N | Time (Days) | 95% Cl |
|---|---|---|---|
| untreated | 5 | n. a.* | |
| VL (POx1); 2 | 30 | 17 | 10 to n. a. |
| ML (IPx2); 3 | 15 | 13 | 13 to 21 |
| ML (POx2); 4 | 30 | 15 | 12 to 17 |
| ML (POx1); 5 | 30 | 21 | 17 to n. a. |
| GL (SCx1); 6 | 15 | 12 | 12 to n. a. |
| M/GL (PO/SCx1); 7 | 15 | n. a.* | |
| VH (POx1); 8 | 30 | 16 | 11 to 20 |
| MH (POx1), 9 | 30 | 23 | 12 to n. a. |

*no death occurred in the untreated group and in group 7 (M/GL (PO/SCx1)) until end of study. Therefore, time to 10% death could not be calculated for these groups.

TABLE 5
Cox proportional hazards regression model
showing the effect of prophylactic treatment
on the risk of radiation induced death

| Variable | Hazard ratio | SE | p | 95% Cl |
|---|---|---|---|---|
| ML (IPx2); 3 | 2.48 | 1.90 | 0.236 | 0.55 to 11.16 |
| ML (POx2); 4 | 1.49 | 0.96 | 0.540 | 0.41 to 5.26 |
| ML (POx1); 5 | 0.64 | 0.49 | 0.565 | 0.14 to 2.88 |

Reference group is VL (POx1); 2.

TABLE 6
Cox proportional hazards regression model
showing the effect of therapeutic treatment on
the risk of radiation induced death^{a,b})

| Variable | Hazard ratio | SE | p | from |
|---|---|---|---|---|
| GL (SCx1); 6 | 0.72 | 0.81 | 0.766 | 0.08 to 6.48 |

^{a}Reference group is VL (POx1); 2.
^{b}no irradiation related deaths in group M/GL (PO/SCx1); 7

TABLE 7
Cox proportional hazards regression model
showing the effect of therapeutic treatment on
the risk of radiation induced death.

| Variable | Hazard ratio | SE | p | 95% Cl |
|---|---|---|---|---|
| MH (POx1); 9 | 0.32 | 0.211 | 0.086 | 0.09 to 1.17 |

Reference group is VH (POx1); 8.

TABLE 8
Survival after 30 days and dose reduction factor for
untreated, vehicle, prophylactic and therapeutic
treatments based on Kaplan-Meier estimates.

| | Radiation | | Quantity ratio of |
|---|---|---|---|
| Group | dose (Gy) | Survival | survival probability * |
| Untreated | 0 | 1.00 | n. a. |
| VL (POx1); 2 | 5.8 | 0.81 | n. a. |
| ML (IPx2); 3 | 5.8 | 0.54 | 0.7 |
| ML (POx2); 4 | 5.8 | 0.72 | 0.9 |
| ML (POx1); 5 | 5.8 | 0.85 | 1.1 |
| GL (SCx1); 6 | 5.8 | 0.90 | 1.1 |
| M/GL (PO/SCx1); 7 | 5.8 | 1.00 | 1.2 |
| VH (POx1); 8 | 6 | 0.56 | n. a. |
| MH (POx1); 9 | 6 | 0.86 | 1.5 |

* The survival probabilities in each group were analysed using the Kaplan-Meier method. The treatment effect was defined to be the ratio of the survival probabilities on day 30 of the treated groups relative to the corresponding control group VL (POx1);
2 for 5.8 Gy irradiation and VH (POx1);
8 and for 6.0 Gy irradiation, respectively

TABLE 9
Summary of results

| | | Primary outcome | | | | | | |
|---|---|---|---|---|---|---|---|---|
| | | Quantity | | Supporting variables | | | | |
| | | ratio of | | | Bone | | | |
| | Details of | survival | | | marrow | Testes | ARS | Body |
| | group | probability | Survival | Hematology | celluarity | degradation | scores | weight |
| Group 2 | VL (PO × 1); 2 | n. a.* | 0.81* | n. a. | n. a. | n. a. | n. a. | n. a. |
| Group 3 | ML (IP × 2); 3 | 0.7 | 0.54 | +/0 | + | + | − | +/0 |
| Group 4 | ML (PO × 2); 4 | 0.9* | 0.72* | 0 | −/0 | + | +/0 | +/0 |
| Group 5 | ML (PO × 1); 5 | 1.1* | 0.85* | +/0 | − | + | + | +/0 |
| Group 6 | GL (SC × 1); 6 | 1.1 | 0.90 | +/0 | + | + | + | +/0 |
| Group 7 | M/GL (PO/SC × 1); 7 | 1.2 | 1.00 | +/0 | + | + | + | +/0 |
| Group 8 | VH (PO × 1); 8 | n. a.* | 0.56* | n. a. | n. a. | n. a. | n. a. | n. a. |
| Group 9 | MH (PO × 1); 9 | 1.5* | 0.86* | +/0 | + | + | + | + |
| Group 2 | VL (PO × 1); 2 | n. a.* | 0.81* | n. a. | n. a. | n. a. | n. a. | n. a. |

positive trend (descriptively) +
negative trend (descriptively) −
no difference (descriptively) 0
*Group 2, 4, 5, 8 and 9 had larger number of animals to allow for a more granular mortality endpoint assessment.

TABLE 10
Statistical tests for different posture scores at days 3, 9,
15, 21, and 30 (AM)
Posture

| | | |
|---|---|---|
| group | day | p-value |

Kruskal Wallis Test

| | | |
|---|---|---|
| 2, 3, 4, 5 | 3 | n.a. |
| | 9 | 0.006 |
| | 15 | 0.031 |
| | 21 | 0.568 |
| | 30 | 0.135 |
| 2, 6, 7 | 3 | n.a. |
| | 9 | 0.152 |
| | 15 | 0.004 |
| | 21 | 0.427 |
| | 30 | 0.103 |

Wilcoxon Ranksum test

| | | |
|---|---|---|
| 8, 9 | 3 | n.a. |
| | 9 | 0.914 |
| | 15 | 0.190 |
| | 21 | 0.094 |
| | 30 | 0.401 |

TABLE 11
Statistical tests for different coat scores at
days 3, 9, 15, 21, and 30 (AM)
Coat

| | | |
|---|---|---|
| group | day | p-value |

Kruskal Wallis Test

| | | |
|---|---|---|
| 2, 3, 4, 5 | 3 | n.a. |
| | 9 | 0.475 |
| | 15 | 0.669 |
| | 21 | 0.701 |
| | 30 | 0.851 |
| 2, 6, 7 | 3 | n.a. |
| | 9 | 0.033 |
| | 15 | 0.422 |
| | 21 | 0.163 |
| | 30 | 0.376 |

Wilcoxon Ranksum test

| | | |
|---|---|---|
| 8, 9 | 3 | n.a. |
| | 9 | n.a. |
| | 15 | 0.433 |
| | 21 | 0.031 |
| | 30 | 0.533 |

TABLE 12
Statistical tests for different behavior
scores at days 3, 9, 15, 21, and 30 (AM)
Behavior
Kruskal Wallis Test

| group | day | p-value |
|---|---|---|
| 2, 3, 4, 5 | 3 | n.a. |
| | 9 | n.a. |
| | 15 | 0.078 |
| | 21 | 0.114 |
| | 30 | 0.703 |

Kruskal Wallis Test

| group | day | p-value |
|---|---|---|
| 2, 6, 7 | 3 | n.a. |
| | 9 | n.a. |
| | 15 | n.a. |
| | 21 | n.a. |
| | 30 | 0.741 |

Wilcoxon Ranksum test

| group | day | p-value |
|---|---|---|
| 8, 9 | 3 | n.a. |
| | 9 | n.a. |
| | 15 | 0.344 |
| | 21 | 0.224 |
| | 30 | n.a. |

TABLE 13A
Body weight in vehicle group 2 (VL (PO × 1); 2) on Day 0, change
from baseline in vehicle group on Days 3, 9, 15, 21, 30.

| | | % | | | |
|---|---|---|---|---|---|
| | | Difference | | | |
| | | from | | | |
| | | Baseline | | | |
| Group | Day | (g) | p | 95% Cl | |
| VL (PO × 1); 2 | 0 | 27.6 | <0.001 | 26.9 | 28.2 |
| ML (IP × 2); 3 | 0 | −0.8 | 0.208 | −1.9 | 0.4 |
| ML (PO × 2); 4 | 0 | −0.3 | 0.532 | −1.3 | 0.7 |
| ML (PO × 1); 5 | 0 | −0.3 | 0.559 | −1.2 | 0.7 |
| VL (PO × 1); 2 | 3 | −1.9 | <0.001 | −2.1 | −1.7 |
| VL (PO × 1); 2 | 9 | −1.7 | <0.001 | −2.1 | −1.3 |
| VL (PO × 1); 2 | 15 | −1.9 | <0.001 | −2.9 | −0.9 |
| VL (PO × 1); 2 | 21 | −3.7 | <0.001 | −5.1 | −2.2 |
| VL (PO × 1); 2 | 30 | −1.4 | <0.001 | −3.0 | 0.2 |

TABLE 13B
Body weight in vehicle group 2 (VL (PO × 1); 2) on Day 0, changes in
Groups 3 (ML (IP × 2); 3), 4 (ML (PO × 2); 4) and 5 (ML
(PO × 1); 5) relative to vehicle group on Days 0, 3, 9, 15, 21, 30.

| | | % | | | |
|---|---|---|---|---|---|
| | | Difference | | | |
| | | from | | | |
| Group | Day | Vehicle(g) | p | 95% Cl | |
| VL (PO × 1); 2 | 0 | 27.6 | <0.001 | 26.9 | 28.2 |
| ML (IP × 2); 3 | 3 | 0.6 | 0.002 | 0.2 | 1.0 |
| ML (IP × 2); 3 | 9 | 0.5 | 0.182 | −0.3 | 1.3 |
| ML (IP × 2); 3 | 15 | 0.1 | 0.930 | −1.9 | 2.1 |
| ML (IP × 2); 3 | 21 | 1.0 | 0.521 | −2.0 | 4.0 |
| ML (IP × 2); 3 | 30 | 0.1 | 0.972 | −3.4 | 3.5 |
| ML (PO × 2); 4 | 3 | 0.7 | <0.001 | 0.4 | 1.0 |
| ML (PO × 2); 4 | 9 | 0.6 | 0.037 | 0.0 | 1.2 |
| ML (PO × 2); 4 | 15 | −0.8 | 0.278 | −2.1 | 0.6 |
| ML (PO × 2); 4 | 21 | −0.7 | 0.494 | −2.8 | 1.3 |
| ML (PO × 2); 4 | 30 | −1.1 | 0.353 | −3.3 | 1.2 |
| ML (PO × 1); 5 | 3 | 0.4 | 0.015 | 0.1 | 0.7 |
| ML (PO × 1); 5 | 9 | 0.4 | 0.151 | −0.2 | 1.0 |
| ML (PO × 1); 5 | 15 | −0.4 | 0.526 | −1.8 | 0.9 |
| ML (PO × 1); 5 | 21 | −0.1 | 0.932 | −2.1 | 1.9 |
| ML (PO × 1); 5 | 30 | 0.1 | 0.937 | −2.1 | 2.2 |

TABLE 13C
Body weight in vehicle group 2 (VL (PO × 1); 2) on Day 0, change from baseline in
vehicle group on Days 3, 9, 15, 21, 30 and changes in Groups 6 (GL (SC × 1); 6) and
7 (M/GL (PO/SC × 1); 7) relative to vehicle group on Days 0, 3, 9, 15, 21, 30.

| Group | Day | Estimate | p | 95% Cl | |
|---|---|---|---|---|---|
| VL (PO × 1); 2 | 0 | 27.6 | <0.001 | 27.0 | 28.1 |
| GL (SC × 1); 6 | 0 | −0.5 | 0.312 | −1.5 | 0.5 |
| M/GL (PO/SC × 1); 7 | 0 | −0.7 | 0.173 | −1.6 | 0.3 |
| VL (PO × 1); 2 | 3 | −1.9 | <0.001 | −2.1 | −1.7 |
| VL (PO × 1); 2 | 9 | −1.7 | <0.001 | −2.3 | −1.1 |
| VL (PO × 1); 2 | 15 | −2.0 | <0.001 | −2.7 | −1.3 |
| VL (PO × 1); 2 | 21 | −4.3 | <0.001 | −5.6 | −3.0 |
| VL (PO × 1); 2 | 30 | −2.2 | <0.001 | −4.0 | −0.5 |
| GL (SC × 1); 6 | 3 | 0.3 | 0.128 | −0.1 | 0.7 |
| GL (SC × 1); 6 | 9 | −0.4 | 0.509 | −1.5 | 0.7 |
| GL (SC × 1); 6 | 15 | 0.4 | 0.566 | −1.0 | 1.9 |
| GL (SC × 1); 6 | 21 | 3.3 | 0.014 | 0.7 | 5.9 |
| GL (SC × 1); 6 | 30 | 0.5 | 0.790 | −2.9 | 3.8 |
| M/GL (PO/SC × 1); 7 | 3 | 0.2 | 0.416 | −0.2 | 0.5 |
| M/GL (PO/SC × 1); 7 | 9 | 0.5 | 0.420 | −0.7 | 1.6 |
| M/GL (PO/SC × 1); 7 | 15 | −1.0 | 0.142 | −2.4 | 0.3 |
| M/GL (PO/SC × 1); 7 | 21 | 2.8 | 0.028 | 0.3 | 5.3 |
| M/GL (PO/SC × 1); 7 | 30 | 2.4 | 0.157 | −0.9 | 5.7 |

TABLE 13D
Body weight in vehicle Group 8 (VH (PO × 1); 8) on Days 0, change from
baseline in vehicle group on Days 3, 9, 15, 21, and 30 and changes in 9
(MH (PO × 1); 9) relative to vehicle on Days 0, 3, 9, 15, 21, and 30.

| | | Estimate | | | |
|---|---|---|---|---|---|
| Group | Day | (g) | p | 95% Cl | |
| VH (PO × 1); 8 | 0 | 27.7 | <0.001 | 27.0 | 28.3 |
| MH (PO × 1); 9 | 0 | 0.0 | 0.983 | −0.9 | 0.9 |
| VH (PO × 1); 8 | 3 | −2.4 | <0.001 | −2.7 | −2.2 |
| VH (PO × 1); 8 | 9 | −1.9 | <0.001 | −2.2 | −1.6 |
| VH (PO × 1); 8 | 15 | −4.0 | <0.001 | −5.0 | −3.1 |
| VH (PO × 1); 8 | 21 | −6.2 | <0.001 | −7.7 | −4.7 |
| VH (PO × 1); 8 | 30 | −3.2 | <0.001 | −4.4 | −2.0 |
| MH (PO × 1); 9 | 3 | 0.2 | 0.282 | −0.2 | 0.6 |
| MH (PO × 1); 9 | 9 | 0.4 | 0.067 | 0.0 | 0.9 |
| MH (PO × 1); 9 | 15 | 1.4 | 0.053 | 0.0 | 2.7 |
| MH (PO × 1); 9 | 21 | 3.1 | 0.006 | 0.9 | 5.2 |
| MH (PO × 1); 9 | 30 | 2.6 | 0.001 | 1.0 | 4.3 |

TABLE 14
Statistical tests severity of testes
degeneration at days 7, 14, 30
Testes

| group | day | p-value |
|---|---|---|
| Kruskal Wallis Test | | |
| 2, 3, 4, 5 | 7 | 0.392 |
| | 14 | 0.037 |
| | 30 | n. a. |
| 2, 6, 7 | 7 | n. a. |
| | 14 | 0.078 |
| | 30 | n.a. |
| Wilcoxon Ranksum | | |
| test | | |
| 8, 9 | 7 | n. a. |
| | 14 | 0.264 |
| | 30 | n. a. |

TABLE 15
Statistical tests for decrease of bone marrow cellularity
in the sternum and femur at days 7, 14, 30

| Sternum | | | Femur | | |
|---|---|---|---|---|---|
| Kruskal Wallis | | | Kruskal Wallis | | |
| Test | | | Test | | |
| group | day | p | group | day | p-value |
| 2, 3, 4, 5 | 7 | n.a. | 2, 3, 4, 5 | 7 | n.a. |
| | 14 | 0.500 | | 14 | 0.492 |
| | 30 | 0.404 | | 30 | 0.001 |
| 2, 6, 7 | 7 | n.a. | 2, 6, 7 | 7 | n.a. |
| | 14 | 0.472 | | 14 | 0.311 |
| | 30 | 0.390 | | 30 | 0.735 |
| Wilcoxon Ranksum test | | | Wilcoxon Ranksum test | | |
| 8, 9 | 7 | n.a. | 8, 9 | 7 | n.a. |
| | 14 | 0.120 | | 14 | 0.371 |
| | 30 | 0.653 | | 30 | 0.197 |

1. A method for treatment of radiation-induced damage, comprising administering to a subject in need thereof a composition comprising imidazolyl ethanamide pentandioic acid.

2. The method according to claim 1, wherein the imidazolyl ethanamide pentandioic acid is administered to a human patient at a dose of 0.4 mg/kg to 12 mg/kg b.w., particularly 1.2 mg/kg to 12 mg/kg b.w., more particularly 4 mg/kg to 12 mg/kg b.w.

3. The method according to claim 1, wherein the imidazolyl ethanamide pentandioic acid is administered to a patient of age 2 to 16 at a dose of 0.5 mg/kg b.w. to 3.75 mg/kg b.w., particularly at a dose of 1.25 mg/kg b.w. twice a day.

4. The method according to claim 1, wherein the imidazolyl ethanamide pentandioic acid is administered at 1 h to 120 h before radiation exposure, particularly 1 h to 72 h before radiation exposure, more particularly at 6 h to 48 h before radiation exposure, most particularly at 12 h to 24 h before radiation exposure.

5. The method according to claim 1, wherein a first dose of imidazolyl ethanamide pentandioic acid is administered at 24 h to 120 h after radiation exposure, particularly 24 h to 72 h after radiation exposure, more particularly at 24 h to 48 h after radiation exposure.

6. The method according to claim 1, wherein a first dose of imidazolyl ethanamide pentandioic acid is administered at 72 h to 6 h after radiation exposure, particularly at 48 h to 8 h after radiation exposure, more particularly at 24 h to 12 h after radiation exposure.

7. The method according to claim 1, wherein the radiation-induced damage results from a radiation dose of
between 0.2 Gy and 35 Gy of total body irradiation, particularly between 0.2 Gy and 13.5 Gy of total body irradiation,
between 0.2 Gy and 4.0 Gy of daily total body radiation,
between 20 Gy and 80 Gy of focal radiation, or
between 1.8 Gy and 30 Gy of daily focal radiation, particularly between 1.8 Gy and 2.0 Gy of daily focal radiation.

8. The method according to claim 1, wherein the radiation-induced damage results from a radiation dose of
between 1.5 Gy and 30 Gy of daily focal radiation, particularly between 1.5 and 1.8 Gy of daily focal radiation
in a patient of age 2 to 17, particularly 2 to 16, more particularly 3 to 16 (pediatric radiation therapy).

9. The method according to claim 1, wherein the subject is exposed to radiation at an acute lethal or near lethal dose sufficient to generate symptoms associated with acute radiation syndrome (ARS) or delayed effects of acute radiation exposure (DEARE).

10. The method according to claim 1, wherein the radiation-induced damage is caused by radiation therapy, by radioisotope contamination (e.g. accidental leak of a nuclear reactor), by chronic low dose cosmic radiation or by the radiation of a nuclear weapon, particularly by radiation therapy in cancer treatment.

11. The method according to claim 1, wherein the imidazolyl ethanamide pentandioic acid is administered orally, intraperitoneally and intravenously, particularly orally.

12. The method according to claim 1, wherein the imidazolyl ethanamide pentandioic acid is administered daily for at least three days, particularly five to ten days.

13. The method according to claim 1, wherein the imidazolyl ethanamide pentandioic acid is administered daily for at least three days post radiation exposure, particularly five to ten days post radiation exposure.

14. The method according to claim 1, wherein the radiation-induced damage is caused by ionizing radiation, particularly by photon radiation.

15. The method according to claim 1, wherein the treatment modality comprises external-beam radiation therapy, particularly the external-beam radiation therapy is selected from the group consisting of intensity-modulated radiation therapy (IMRT), image-guided radiation therapy (IGRT), tomotherapy, stereotactic radiosurgery, stereotactic body radiation therapy, photon beam, electron beam and proton or neutron therapy.

16. The method according to claim 1, wherein the radiation-induced damage results from a radiation therapy comprising
a. internal radiation therapy or brachytherapy, or
b. systemic radiation therapy.

17. The method according to claim 1, wherein the imidazolyl ethanamide pentandioic acid is administered in combination with G-CSF, GM-CSF, peg-G-CSF or peg-GM-CSF, particularly wherein G-CSF, GM-CSF, lipeg-G-CSF, peg-G-CSF or peg-GM-CSF is administered at a dose of 2 μg/kg b.w./day to 30 μg/kg b.w./day, particularly at a dose of 2.8 μg/kg b.w./day to 10 μg/kg b.w./day.

18. A combination medicament for use in treatment or prevention of radiation-induced or chemotherapy-induced damage, said combination medicament comprising
imidazolyl ethanamide pentandioic acid and
G-CSF, GM-CSF, peg-G-CSF or peg-GM-CSF.
